SEQUENCE LISTING (78 sequences)

SEQ ID NO: 1 - DNA, 22 bp, Artificial Sequence
  Probe
cagagagcttgctctcgggtga

SEQ ID NO: 2 - DNA, 26 bp, Artificial Sequence
  Probe
gggaggaaggtgttgtggttaataac

SEQ ID NO: 3 - DNA, 26 bp, Artificial Sequence
  Probe
ggtgttgtggttaataaccacagcaa

SEQ ID NO: 4 - DNA, 22 bp, Artificial Sequence
  Probe
gcggtctgtcaagtcggatgtg

SEQ ID NO: 5 - DNA, 25 bp, Artificial Sequence
  Probe
attcgaaactggcaggctagagtct

SEQ ID NO: 6 - DNA, 25 bp, Artificial Sequence
  Probe
taaccacagcaattgacgttacccg

SEQ ID NO: 7 - DNA, 24 bp, Artificial Sequence
  Probe
gcaattgacgttacccgcagaaga

SEQ ID NO: 8 - DNA, 26 bp, Artificial Sequence
  Probe
gaaccgcatggttcaaaagtgaaaga

SEQ ID NO: 9 - DNA, 24 bp, Artificial Sequence
  Probe
cacttatagatggatccgcgctgc

SEQ ID NO: 10 - DNA, 26 bp, Artificial Sequence
  Probe
tgcacatcttgacggtacctaatcag

SEQ ID NO: 11 - DNA, 22 bp, Artificial Sequence
  Probe
ccccttagtgctgcagctaacg

SEQ ID NO: 12 - DNA, 23 bp, Artificial Sequence
  Probe
aatacaaagggcagcgaaaccgc

SEQ ID NO: 13 - DNA, 25 bp, Artificial Sequence
  Probe
ccggtggagtaaccttttaggagct

SEQ ID NO: 14 - DNA, 25 bp, Artificial Sequence
  Probe
taaccttttaggagctagccgtcga

SEQ ID NO: 15 - DNA, 23 bp, Artificial Sequence
  Probe
tttaggagctagccgtcgaaggt

SEQ ID NO: 16 - DNA, 23 bp, Artificial Sequence
  Probe
tagccgtcgaaggtgggacaaat

SEQ ID NO: 17 - DNA, 22 bp, Artificial Sequence
  Probe
gaacagacgaggagcttgctcc

SEQ ID NO: 18 - DNA, 26 bp, Artificial Sequence
  Probe
tagtgaaagacggttttgctgtcact

SEQ ID NO: 19 - DNA, 26 bp, Artificial Sequence
  Probe
taagtaactatgcacgtcttgacggt

SEQ ID NO: 20 - DNA, 26 bp, Artificial Sequence
  Probe
gacccctctagagatagagttttccc

SEQ ID NO: 21 - DNA, 24 bp, Artificial Sequence
  Probe
agtaaccatttggagctagccgtc

SEQ ID NO: 22 - DNA, 23 bp, Artificial Sequence
  Probe
gagcttgctcctctgacgttagc

SEQ ID NO: 23 - DNA, 22 bp, Artificial Sequence
  Probe
agccggtggagtaaccatttgg

SEQ ID NO: 24 - DNA, 22 bp, Artificial Sequence
  Probe
ctcttgccatcggatgtgccca

SEQ ID NO: 25 - DNA, 26 bp, Artificial Sequence
  Probe
atacctttgctcattgacgttacccg

SEQ ID NO: 26 - DNA, 24 bp, Artificial Sequence
  Probe
tttgctcattgacgttacccgcag

SEQ ID NO: 27 - DNA, 24 bp, Artificial Sequence
  Probe
actggcaagcttgagtctcgtaga

SEQ ID NO: 28 - DNA, 23 bp, Artificial Sequence
  Probe
atacaaagagaagcgacctcgcg

SEQ ID NO: 29 - DNA, 25 bp, Artificial Sequence
  Probe
cggacctcataaagtgcgtcgtagt

SEQ ID NO: 30 - DNA, 26 bp, Artificial Sequence
  Probe
gcggggaggaagggagtaaagttaat

SEQ ID NO: 31 - DNA, 23 bp, Artificial Sequence
  Probe
tagcacagagagcttgctctcgg

SEQ ID NO: 32 - DNA, 23 bp, Artificial Sequence
  Probe
tcatgccatcagatgtgcccaga

SEQ ID NO: 33 - DNA, 25 bp, Artificial Sequence
  Probe
cggggaggaaggcgataaggttaat

SEQ ID NO: 34 - DNA, 25 bp, Artificial Sequence
  Probe
ttcgattgacgttacccgcagaaga

SEQ ID NO: 35 - DNA, 26 bp, Artificial Sequence
  Probe
ggtctgtcaagtcggatgtgaaatcc

SEQ ID NO: 36 - DNA, 24 bp, Artificial Sequence
  Probe
gcaggctagagtcttgtagagggg

SEQ ID NO: 37 - DNA, 24 bp, Artificial Sequence
  Probe
tgagggagaaagtgggggatcttc

SEQ ID NO: 38 - DNA, 25 bp, Artificial Sequence
  Probe
tcagatgagcctaggtcggattagc

SEQ ID NO: 39 - DNA, 24 bp, Artificial Sequence
  Probe
gagctagagtacggtagagggtgg

SEQ ID NO: 40 - DNA, 25 bp, Artificial Sequence
  Probe
gtacggtagagggtggtggaatttc

SEQ ID NO: 41 - DNA, 24 bp, Artificial Sequence
  Probe
gaccacctggactgatactgacac

SEQ ID NO: 42 - DNA, 25 bp, Artificial Sequence
  Probe
tggccttgacatgctgagaactttc

SEQ ID NO: 43 - DNA, 23 bp, Artificial Sequence
  Probe
ttagttaccagcacctcgggtgg

SEQ ID NO: 44 - DNA, 22 bp, Artificial Sequence
  Probe
tagtctaaccgcaagggggacg

SEQ ID NO: 45 - DNA, 22 bp, Artificial Sequence
  Probe
tagcacagggagcttgctccct

SEQ ID NO: 46 - DNA, 25 bp, Artificial Sequence
  Probe
aggtggtgagcttaatacgctcatc

SEQ ID NO: 47 - DNA, 26 bp, Artificial Sequence
  Probe
tcatcaattgacgttactcgcagaag

SEQ ID NO: 48 - DNA, 26 bp, Artificial Sequence
  Probe
actgcatttgaaactggcaagctaga

SEQ ID NO: 49 - DNA, 24 bp, Artificial Sequence
  Probe
ttatcctttgttgcagcttcggcc

SEQ ID NO: 50 - DNA, 23 bp, Artificial Sequence
  Probe
actttcagcgaggaggaaggtgg

SEQ ID NO: 51 - DNA, 25 bp, Artificial Sequence
  Probe
agtagaacgctgaaggaggagcttg

SEQ ID NO: 52 - DNA, 25 bp, Artificial Sequence
  Probe
cttgcatcactaccagatggacctg

SEQ ID NO: 53 - DNA, 26 bp, Artificial Sequence
  Probe
tgagagtggaaagttcacactgtgac

SEQ ID NO: 54 - DNA, 26 bp, Artificial Sequence
  Probe
gctgtggcttaaccatagtaggcttt

SEQ ID NO: 55 - DNA, 23 bp, Artificial Sequence
  Probe
aagcggctctctggcttgtaact

SEQ ID NO: 56 - DNA, 24 bp, Artificial Sequence
  Probe
tagaccctttccggggtttagtgc

SEQ ID NO: 57 - DNA, 26 bp, Artificial Sequence
  Probe
gacggcaagctaatctcttaaagcca

SEQ ID NO: 58 - DNA, 24 bp, Artificial Sequence
  Probe
gcttgggaatctggcttatggagg

SEQ ID NO: 59 - DNA, 23 bp, Artificial Sequence
  Probe
tgccataggatgagcccaagtgg

SEQ ID NO: 60 - DNA, 26 bp, Artificial Sequence
  Probe
cttgggaatgtactgacgctcatgtg

SEQ ID NO: 61 - DNA, 23 bp, Artificial Sequence
  Probe
ggattgggcttagagcttggtgc

SEQ ID NO: 62 - DNA, 22 bp, Artificial Sequence
  Probe
tacagagggaagcgaagctgcg

SEQ ID NO: 63 - DNA, 26 bp, Artificial Sequence
  Probe
ggcgtttaccacggtatgattcatga

SEQ ID NO: 64 - DNA, 23 bp, Artificial Sequence
  Probe
aatgcctaccaagcctgcgatct

SEQ ID NO: 65 - DNA, 25 bp, Artificial Sequence
  Probe
tatcggaagatgaaagtgcgggact

SEQ ID NO: 66 - DNA, 23 bp, Artificial Sequence
  Probe
ttctttcctcccgagtgcttgca

SEQ ID NO: 67 - DNA, 25 bp, Artificial Sequence
  Probe
aacacgtgggtaacctacccatcag

SEQ ID NO: 68 - DNA, 24 bp, Artificial Sequence
  Probe
atggcataagagtgaaaggcgctt

SEQ ID NO: 69 - DNA, 22 bp, Artificial Sequence
  Probe
gacccgcggtgcattagctagt

SEQ ID NO: 70 - DNA, 25 bp, Artificial Sequence
  Probe
ggacgttagtaactgaacgtcccct

SEQ ID NO: 71 - DNA, 22 bp, Artificial Sequence
  Probe
ctcaaccggggagggtcattgg

SEQ ID NO: 72 - DNA, 22 bp, Artificial Sequence
  Probe
ttggagggtttccgcccttcag

SEQ ID NO: 73 - DNA, 23 bp, Artificial Sequence
  Primer
gcggcgtgcctaatacatgcaag

SEQ ID NO: 74 - DNA, 23 bp, Artificial Sequence
  Primer
gcggcaggcctaacacatgcaag

SEQ ID NO: 75 - DNA, 23 bp, Artificial Sequence
  Primer
gcggcaggcttaacacatgcaag

SEQ ID NO: 76 - DNA, 22 bp, Artificial Sequence
  Primer
atccagccgcaccttccgatac

SEQ ID NO: 77 - DNA, 22 bp, Artificial Sequence
  Primer
atccaaccgcaggttcccctac

SEQ ID NO: 78 - DNA, 22 bp, Artificial Sequence
  Primer
atccagccgcaggttcccctac

(19) United States
(12) Patent Application Publication
(10) Pub. No.: US 2010/0296711 A1
(43) Pub. Date: Nov. 25, 2010

(54) METHOD OF DETERMINING A BIOSPECIES

(75) Inventor: Hiroto Yoshii, Tokyo (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 11/996,744

(22) PCT Filed: Aug. 4, 2006
(86) PCT No.: PCT/JP2006/315898

§ 371 Date: Jan. 24, 2008

(30) Foreign Application Priority Data
Aug. 5, 2005 (JP) 2005-227995

(51) Int. Cl. C12Q 1/68; G06K 9/00

(52) U.S. Cl. 382/129; 435/6

(57) ABSTRACT

Determining a biospecies is performed by using a plurality of analysis data obtained by analyzing a plurality of known samples whose corresponding biospecies are already revealed by a method of analyzing an organism and a determination threshold defined on the basis of the plurality of analysis data; deciding whether determination of a biospecies corresponding to an unknown sample is possible or not on the basis of the determination threshold; and determining a biospecies corresponding to the unknown sample on the basis of the plurality of analysis data when the determination is decided as possible.

### TECHNICAL FIELD

The present invention relates to a method of determining a biospecies using pattern recognition, particularly one that can be suitably applied to a system for analyzing a nucleic acid sequence using a DNA microarray and can exert its effect when it is used in application of determination of a microbial species.

### BACKGROUND ART

As one of the conventional methods of determining biospecies, a method that utilizes a DNA microarray equipped with nucleic acid fragments referred to as “probe” positioned and immobilized on a substrate made of glass or the like has been known in the art. This method utilizes the DNA microarray to analyze an unknown sample of nucleic acid fragment (hereinafter, simply referred to as “unknown sample”), to thereby determine what biospecies is the unknown sample. In this method, base-pairing reaction, or hybridization reaction, of nucleic acid is employed. Hybridization reaction can be outlined as follows. In most cases within a living body, DNA exists in a double helix structure and the link between two strands thereof is realized by a hydrogen bond between bases. In contrast, mostly, RNA exists in a single strand structure. For DNA, there are four different bases, A, T, G, and C. For RNA, there are four different bases, A, U, G, and C. Among those bases, hydrogen bonds can be formed between the respective pairs of A-T(U) and G-C. Thus, hybridization reaction means that two nucleic acid molecules in a single strand form react with each other under appropriate conditions and then united into one through the base sequences of the nucleic acids.

Based on this fact, hereinafter, the conventional Method of determining a biospecies will be described. A hybridization reaction can occur between a probe immobilized on a substrate and a nucleic acid fragment having a complementary base sequence capable of forming base pairs with the probe under appropriate conditions, thereby allowing the binding of the probe with the nucleic acid fragment. Determination of biospecies may only be achieved on the fact that a probe immobilized on the substrate has a base sequence corresponding to that of a certain organism and the binding of the probe with the nucleic acid fragment is recognized through hybridization reaction. It allows that a biospecies corresponding to the nucleic acid fragment can be identified to be identical with one corresponding to the probe. In other words, any biospecies corresponding to an unknown sample can be determined.

For instance, by providing a nucleic acid fragment with a fluorescent substance, it is possible to optically recognize whether hybridization reaction has occurred. When fluorescence has been produced from the probe immobilized on the substrate, it is recognized that hybridization reaction has occurred to form a hybrid between the probe and the nucleic acid fragment and it is thus determined that the nucleic acid fragment is identical with the biospecies corresponding to the probe. In contrast, when the probe does not generate fluorescence, it is recognized that no hybrid between the probe and the nucleic acid fragment is formed. Therefore, it is determined that the nucleic acid fragment is not of the biospecies corresponding to the probe. Using the above determination method, when an unknown sample is provided, the determination on which of two or more biospecies corresponds to the sample can be carried out by a single hybridization reaction. That is, two or more probes whose corresponding biospecies have been known, are prepared and then immobilized on their respective predetermined positions on the substrate to make a DNA microarray. Then, the DNA microarray thus prepared is subjected to a hybridization reaction with an unknown sample under appropriate conditions. Thus, a biospecies can be identified from the location thereof on the substrate and it is hence determined whether the sample corresponds to the biospecies on the basis of the presence or absence of fluorescence at the location. In other words, by confirming the location on the substrate from which fluorescence is generated, a biospecies corresponding to the unknown sample can be determined.

Actually, however, from the result of the hybridization reaction of an unknown sample, fluorescence generation does not always occur from a probe corresponding to a single kind of biospecies. In many cases, fluorescence may be generated not only,from the intended fluorescence-generating probe but also from another probe when the hybridization reaction is carried out, even though it is known that the unknown sample corresponds to a single kind of biospecies. This is because the nucleic acid molecule of the unknown sample may partially bind to another probe through a certain base sequence in such a molecule. This phenomenon is referred to as “cross-hybridization”. Thus, the generation of cross-hybridization makes it impossible to determine a biospecies corresponding to an unknown sample based on only two pieces of information, the location on the substrate and the presence or absence of fluorescence as described above.

For instance, if the assumption is made that an unknown sample is subjected to a hybridization reaction with a DNA microarray having probes that correspond to their respective kinds of biospecies. The biospecies in the unknown sample will not be determined as biospecies A or biospecies B when probes corresponding to organisms A and B generate fluorescence.

Considering the possibility of cross hybridization, for example, three different cases can be conceivable: the unknown sample only corresponds to organism A; the unknown sample corresponds to only organism B; and the unknown sample corresponds to both organism A and organism B.

As a general tendency, with respect to the intensities of fluorescence generated from nucleic acid fragments binding to the same probe, the fluorescence intensity from an almost completely-hybridized fragment is stronger than the fluorescence intensity from a partially-hybridized fragment by cross hybridization. Therefore, when a DNA microarray is employed to analyze an unknown sample for determining which of biospecies corresponds to the unknown sample, a method of determining a biospecies should be selected from the overall viewpoints of positional information about probes and information about signal intensities represented by fluorescence intensities.

The fluorescence intensity after a hybridization reaction with an unknown sample is stored as vector data having the order of probe locations in a storage means.

In JP-A-2002-533699, there is disclosed a method of retrieving known vector data which is most analogous to the vector data obtained from an unknown sample by analyzing vector date obtained from the unknown sample utilizing a DNA microarray. The information processing, where the most resemble known vector data is retrieved, is known as pattern recognition and well known in the art. The pattern recognition is a process for corresponding an observed pattern to one of previously-defined “categories”.

In the technological field of OCR (Optical Character Recognition), the “categories” can be exemplified by using pattern recognition in which one character printed or hand-written on paper is recognized as one pattern. In this case, if a recognition target is a numeric character, “which of numerals, 0 to 9, has the most resemblance to the numeral written on paper?” is determined by comparing it with the known vector data. In this pattern recognition, “categories” are ten numerals from 0 to 9 to be recognized.

Typically, in the case of the pattern recognition, the number and types of categories to be recognized are previously defined. In the above example, the number and types of categories, for example, 0 to 9 for numeral characters, approximately 3,000 Chinese characters for Japanese, and 26 alphabetical characters for English are previously defined.

### DISCLOSURE OF THE INVENTION

However, when pattern recognition is carried out using vector data obtained by a hybridization reaction of a sample containing a nucleic acid fragment, to which the corresponding biospecies is unknown, with a DNA microarray, categories to be assumed are not always defined in advance. For instance, when a DNA microarray is employed to determine whether a certain bacterial species is present in an unknown sample, the species of bacteria corresponding to a nucleic acid fragment to be provided as a probe must be defined in advance. However, there is a small possibility that the organism, which is actually present in, the unknown sample, is one of the biospecies corresponding to such probes. This is because the number of all kinds of biospecies is extensively larger than the number of categories, nine categories for numerals 0 to 9, 26 categories for alphabets A to Z, or approximately 3,000 categories for Chinese characters in the technical field referred to as “OCR” as described above. Therefore, even if the biospecies to be determined are confined to bacterial species, a large number of categories to be assumed will be required and thus the categories for all kinds of bacteria can be virtually impossible to be defined in advance. Therefore, there is a need to define categories while the number of organisms assumed to be present in an unknown sample is confined to some extent.

Thus, the conventional method used for character recognition in OCR or the like cannot be directly applied to the determination of biospecies. When an organism of which category is not defined in advance is included in an unknown sample, there is a problem of causing an error in determination such that the organism may be forced to correspond to a predetermined category.

It is therefore an object of the present invention to reduce the possibility of causing an error in determination when an organism that does not correspond to any of categories defined in advance in the determination of biospecies utilizing pattern recognition.

According to one aspect of the present invention, a method of determining a biospecies by analyzing a sample, in which a substance derived from an organism is supposed to be included, to determine the biospecies corresponding to the organism, includes the steps of: obtaining a plurality of analysis data by analyzing a plurality of known samples whose corresponding biospecies are already revealed, by a method of analyzing a biospecies; defining a determination threshold with respect to the biospecies corresponding to the known sample on the basis of the plurality of analysis data obtained from the plurality of known samples; obtaining analysis data for specifying a biospecies corresponding to an unknown sample whose corresponding biospecies is unknown, by analyzing the unknown sample by the method of analyzing a biospecies; deciding whether determination of a species corresponding to the unknown sample is possible or impossible on the basis of the determination threshold; and determining the biospecies of the unknown sample on the basis of the plurality of analysis data when the determination is decided as possible.

According to the present invention, when an organism which dose not correspond to any of categories previously defined is included in an unknown sample, it can be judged indeterminable and thus there is an advantage in that the determination can result in an appropriate biospecies. In addition, parameters for judging whether the respective categories corresponding to biospecies are indeterminable or not can be defined, so there is an advantage in that the determination can result in the most appropriate biospecies depending on the biological characteristics of the biospecies.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 shows an example of a method of determining a biospecies of the present invention.

FIG. 2 is a block diagram showing a configuration of an information processing apparatus for carrying out the method of determining a biospecies of the present invention.

FIG. 3 is a diagram illustrating a hybridization reaction.

FIG. 4 shows an experimental procedure using a DNA microarray.

FIG. 5 shows an experimental procedure of a DNA microarray for determination of an infectious disease.

FIGS. 6A and 6B each show an example of image formed of fluorescence intensities after hybridization reactions.

FIG. 7 shows a distribution example of vector data.

FIG. 8 is a diagram illustrating a step of defining an indeterminable level.

FIG. 9 shows a distribution example of a determination index set.

FIG. 10 shows an example of a distance set of two arbitrary samples in the same categories.

FIG. 11 shows an experimental data on a DNA microarray for a Klebsiella pneumoniae sample.

FIG. 12 shows an experimental data on the DNA microarray for the Klebsiella pneumoniae sample.

FIG. 13 shows an experimental data on the DNA microarray for the Klebsiella pneumoniae sample.

FIG. 14 shows an experimental data on the DNA microarray for the Klebsiella pneumoniae sample.

FIG. 15 shows an experimental data on the DNA microarray for the Klebsiella pneumoniae sample.

FIG. 16 shows an experimental data on the DNA microarray for the Klebsiella pneumoniae sample.

FIG. 17 shows an experimental data on the DNA microarray for the Klebsiella pneumoniae sample.

FIG. 18 shows an experimental data on the DNA microarray for the Klebsiella pneumoniae sample.

FIG. 19 shows an experimental data on the DNA microarray for the Klebsiella pneumoniae sample.

FIG. 20 shows an experimental data on the DNA microarray for the Klebsiella pneumoniae sample.

FIG. 21 is a histogram with respect to the distance of each arbitrary pair in 10 samples of Klebsiella pneumoniae.

FIG. 22 shows an experimental data on the DNA microarray for a Serratia marcescens sample.

FIG. 23 shows an experimental data on the DNA microarray for the Serratia marcescens sample.

FIG. 24 shows an experimental data on the DNA microarray for the Serratia marcescens sample.

FIG. 25 shows an experimental data on the DNA microarray for the Serratia marcescens sample.

FIG. 26 shows an experimental data on the DNA microarray for the Serratia marcescens sample.

FIG. 27 shows an experimental data on the DNA microarray for the Serratia marcescens sample.

FIG. 28 shows an experimental data on the DNA microarray for the Serratia marcescens sample.

FIG. 29 shows an experimental data on the DNA microarray for the Serratia marcescens sample.

FIG. 30 shows an experimental data on the DNA microarray for the Serratia marcescens sample.

FIG. 31 shows an experimental data on the DNA microarray for the Serratia marcescens sample.

FIG. 32 is a histogram with respect to a distance of each arbitrary pair of 10 samples of Serratia marcescens.

### BEST MODE FOR CARRYING OUT THE INVENTION

Preferred embodiments of the present invention will now be described in detail in accordance with the accompanying drawings.

A method of determining a biospecies according to the present invention includes a method of creating an identification dictionary and defining a determination threshold by analyzing vector data. In the method of determining a biospecies according to the present invention, at first, vector data obtained by analyzing a sample of a nucleic acid fragment extracted from an organism where a biospecies thereof is proved (hereinafter, referred to as “known sample”) is stored in an external storage means. Then, the vector data obtained by analyzing the known sample is referenced like a dictionary to determine the biospecies of an unknown sample, so the whole of vector data for determination of biospecies, which has been obtained by analyzing the known sample stored in the external storage means, can be referred to as “identification dictionary”.

Next, the vector data stored as an identification dictionary is used to define a determination threshold. The details of how to define the determination threshold will be described in detail later. On the basis of the defined determination threshold, a judgment is made on whether an unknown sample whose corresponding biospecies is expected to be revealed can be allowed to determine the biospecies thereof with the created identification dictionary or not (indeterminable).

Hereinafter, the determination method of the present invention will be described in the case of the results obtained by analyzing a known sample and an unknown sample are obtained as image data.

For selecting a known sample for creating an identification dictionary, at first, a biospecies to which an organism provided as a target of the determination is supposed to belong is selected. For instance, if there is a possibility of the existence of bacteria in an unknown sample and the determination of a biospecies for the bacteria is desired to be carried out, any known biospecies may be previously selected from the bacteria. The selected biospecies may correspond to a category in a method of determining a biospecies, which utilizes pattern recognition. We have already mentioned that categories are required to be defined to some extent because the number of the whole biospecies is extremely larger than those of numerals and alphabets.

Next, an individual of each biospecies thus selected is prepared and a sample of a nucleic acid fragment extracted from the individual of each biospecies is then obtained. This sample is provided as a known sample and an analysis method of obtaining image data from the known and unknown samples is then selected. This analysis method is selected from methods that enable the determination of biospecies with pattern recognition. For instance, an analysis method that recognizes an obtained image data as vector data by using a DNA microarray or the like can be suitably used.

An explanation is now given to how to obtain image data using a DNA microarray. Probes are prepared for the respective biospecies and immobilized on predetermined positions on a substrate as described above (i.e., positions on which probes are located on previously-defined positions corresponding to the respective biospecies).

It is possible to optically recognize whether a hybridization reaction has occurred when a DNA microarray is allowed to react with a nucleic acid fragment under appropriate conditions by providing a nucleic acid fragment with a fluorescent substance or the like.

In the present invention, the determination of biospecies can be carried out by defining a determination threshold on the basis of image data obtained from two or more known samples in each category (i.e., two or more different individuals of the same organism).

By the way, when the organism to be determined is a microorganism, the “species” of the microorganism can be selected as a biospecies and it goes without saying that the present invention can be applied on various kinds of other organisms.

Hereinafter, an example of the present invention will be described with reference to the drawings.

FIG. 1 is a flow chart for illustrating a processing procedure in an example of the method of determining a biospecies of the present invention. This method of determining a biospecies is a method of determining whether any substance derived from a biospecies, which can specify the biospecies as a target, resides in a certain unknown sample and for determining, if it resides therein, what kind of the species the organism derived from the biospecies belongs to. The rejection in the method of determining a biospecies is to determine the absence of the substance derived from the biospecies selected as a target in the unknown sample. In the following description, by the way, the present invention will be described such that the determination of a biospecies using a genomic analysis for a microorganism or the like is provided as a subject matter. However, for example, the technology of the present invention can also be applied to an examination system using an antigen-antibody reaction. In addition, the technology of the present invention may be applied in any system that analyzes a genome region or the like for individual recognition, such as MHC.

The flow of the process for determining the biospecies of an unknown sample of the present invention can be mainly divided into a learning phase in which an identification dictionary is created using a known sample and a determination phase in which the unknown sample is determined. In FIG. 1, the learning phase is from 101 to 104 and the determination phase is from 105 to 108.

Hereinafter, the learning phase will be described. In Step 101, a known sample, which contains a nucleic acid fragment extracted from an organism whose corresponding species is known, is prepared. For example, the known sample may be a solution containing the genome of a bacterium whose bacterial species has been specified. A series of steps of a hybridization reaction experiment 102 is carried out using the known sample to obtain data. For instance, when a DNA microarray is employed but the details thereof will be described later, a nucleic acid fragment in the known sample is amplified by a PCR reaction at first and then provided with a fluorescent substance. Subsequently, the nucleic acid fragment is subjected to a hybridization reaction with the DNA microarray. The data on fluorescence intensities of the respective spots is then recognized as an image and stored in an external storage means. On the basis of the image, a determination threshold is defined in a step of defining a determination threshold 103, and an identification dictionary is created in a step of creating a dictionary 104.

Next, the determination phase will be described. An unknown sample is prepared (Step 105) and then a hybridization reaction experiment 106 is carried out just by the same procedure as that of Step 102. Image data obtained from the results, of the reaction is compared with the determination threshold and the identification dictionary each of which is obtained in the learning phase to determine a biospecies with respect to the unknown sample (Step 107).

Consequently, a determination result 108 can be obtained as any of those including: “the unknown sample corresponds to Biospecies A”, “the unknown sample contains substances derived from Biospecies A to C”, “the unknown sample retains a substance derived from an organism other than Biospecies A to Z but included in Biological group α”, and “the unknown sample at 105 cannot be determined (i.e., indeterminable)”.

Hereinafter, specifically with respect to the method of defining a determination threshold in the learning phase as described above, two different methods will be described in detail.

First, known samples obtained from different individuals of the same organism species are prepared and each of them is then subjected to a hybridization reaction with a DNA microarray to obtain image data. For defining a determination threshold, any of the following methods can be preferably used.

Method (1), where one of image data on three or more known samples is chosen and removed and the image data on the remaining known samples is then used to create an identification dictionary, followed by definition of a determination threshold using the identification dictionary.

Method (2), where distances for all of arbitrary combinations of two of image data on three or more known samples are calculated by means of pattern recognition algorithms and then used to define a determination threshold.

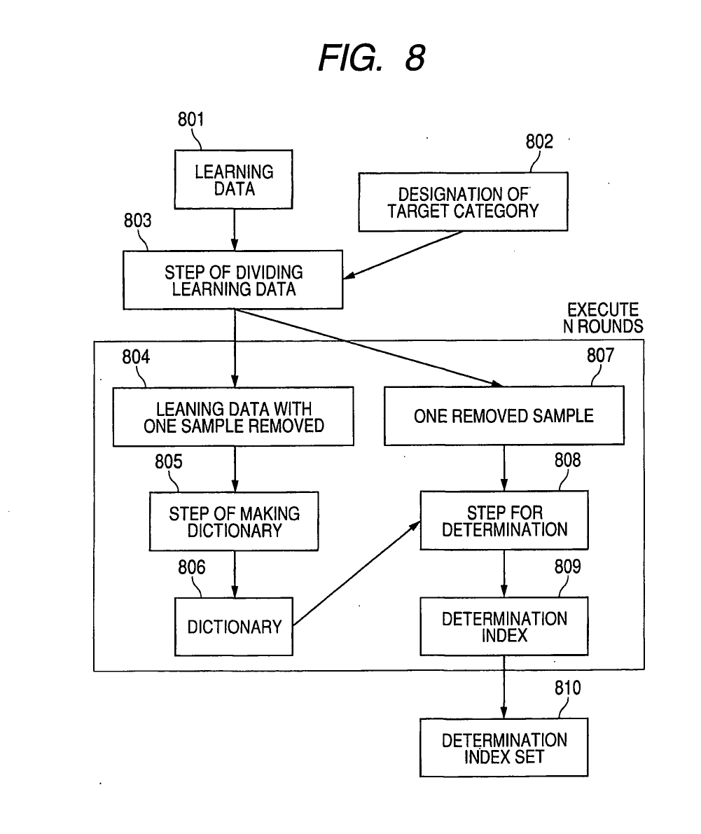

First, the above method (1), the Method of defining a determination threshold, will be described. A flow chart of a processing procedure of the method is shown in FIG. 8. A predetermined number “n” of different biospecies to be supposed as the results of the determination of biospecies (S1 to Sn: n≧2) for an unknown sample, i.e., target categories, are selected (Step 802). Then, each of the target categories thus selected is processed for obtaining a determination threshold inherent therein. Subsequently, a known sample corresponding to the category selected as a target category in Step 802 is prepared and subjected to hybridization, thereby obtaining image data. For creating an identification dictionary, the image data is stored in an external storage means. The whole of the image data is referred to as “learning data” 801. Hereinafter, by exemplifying a biospecies belonging to a target category S1, the above method (1), the method of defining a determination threshold, will be described.

First, “m” individuals S1-X (1≦X≦m, m≧3) belonging to the target category S1 are prepared.

Then, a nucleic acid fragment is extracted from each of the individuals thus prepared to obtain “m” known samples (m≧3). The “m” known samples are subjected to hybridization with a DNA microarray under suitable conditions to obtain an assembly of “m” image data (m>3) (Ps1-1 to Ps1-m).

Next, in the step 803 of dividing learning data, one of the image data is chosen and then removed from the learning data. Subsequently, the remaining “m-1” learning data 804 other than the removed one image data are used to create an identification dictionary 806 in the step 805 of creating a dictionary. The dictionary-creating step 805 makes the dictionary, on the basis of pattern recognition algorithms employed.

For the determination of unknown patterns with pattern recognition, any method selected from methods known by persons skilled in the art can be employed. Methods for determination and categorization with pattern recognition include those reviewed in the article of Anil K. Jain, Robert P. W. Duin, and Jianchan Mao, “Statistical Pattern Recognition: A Review” in IEEE Transaction on Pattern Analysis and Machine Learning, Vol. 22, No. 1, January 2000, pp. 4-37. To be specific, pattern recognition techniques such as k-Nearest-Neighbor, categorization trees, support vector machine, Bayes discrimination, boosting, and neural networks can be utilized.

For instance, when a neural network is employed as a pattern recognition algorithm, an assembly of weighted parameters of the neural network is learned as an identification network. In addition, if the Support Vector Machine is employed as a pattern recognition algorithm, a representative sample vector, which is the so-called Support Vector, and weighting thereof are learned as an identification dictionary. In the present invention, the term “learned as an identification dictionary” or “learns” is synonymous with the creation of an identification dictionary on the basis of learning data.

Next, the one image data removed from the learning data is determined using the identification dictionary 806 (Step 808). Here, for example, it is assumed that the image data Ps 1-1 corresponding to an individual S1-1 is removed from the learning data. At this time, a point to notice is that the identification dictionary 806 does not contain the one image data removed in Step 807. Thus, the identification dictionary 806 considers the individual S1-1 removed in Step 807 to be an unknown sample. For carrying out determination with the identification dictionary, there is a need to define a norm represented by a Euclid norm for making a comparison between vector data stored in an external storage means.

The method of determining a biospecies of the present invention may employ any of various general norms, and the case where a Euclid norm is employed will be described later.

As a result, a determination index 809 can be obtained (Step 809).

In general, the results of determination with pattern recognition algorithms are generally represented by numeral data. For instance, the results may be determination probabilities, similarities, and simply distances between vector data. Thus, the determination index 809 means numeral data obtained as determination results calculated using a previously defined norm.

Therefore, an identification dictionary created using learning data obtained by removing one image data from “m” image data is used to obtain one determination index A1-1 for the target, category S1.

Next, additional removal of the image data on the target category S1 selected from S1-X (1≦X≦m, m≧3), which have not been removed in the step of dividing the learning data 803, is carried out by the same way as described above. Here, it is supposed that image data corresponding to S1-2 is selected. The same processing as described above is carried out to obtain a determination index A1-2 for the target category S1. In other words, by carrying out the same procedures as described above on each image data on the target category S1, a determination index set {A1} consisting of “m” determination indices is obtained. The index set {A1} consists of elements of “m” determination indices, A1-1, A-2, . . . , and A1-m.

A determination threshold for the target category S1 can be defined on the basis of the determination index set {A1} thus obtained. In the above description, the method of obtaining a determination index set has been described while exemplifying the target category S1. Likewise, determination index sets for target categories other than the target category S1 out of “n” different target categories chosen first are also obtained. As a result, “n” determination index sets can be obtained.

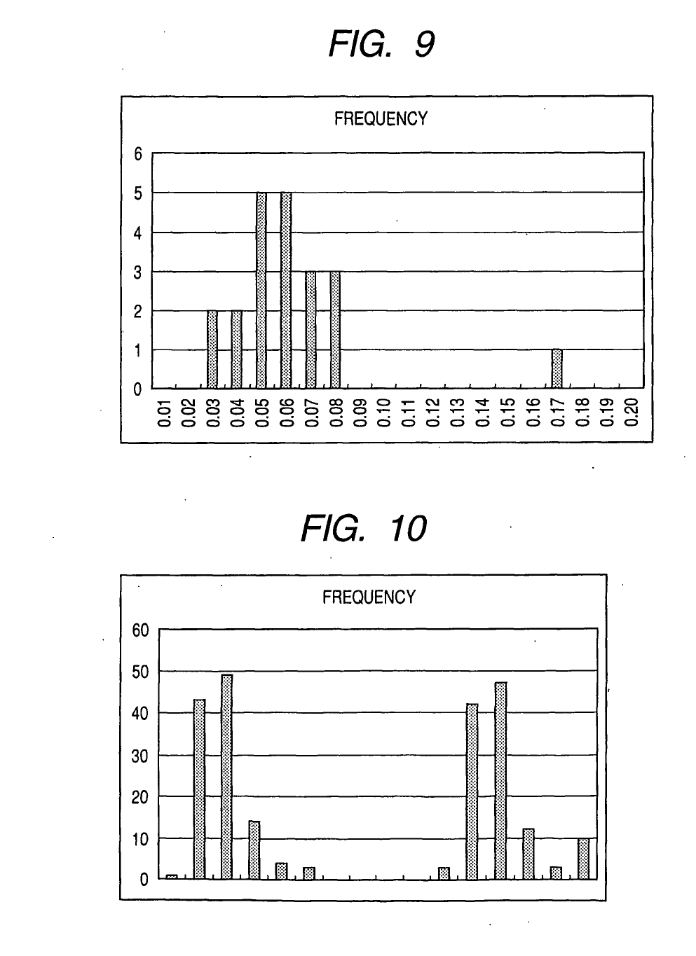

Referring now to FIG. 9, there is shown an example in which one determination index set is selected from “n” determination index sets and the distribution of a determination index set 810 is represented by a histogram. When a determination index 809 shows similarity, a determination threshold is adjusted to “α” folds of the minimum value of the set (α<1) or “β” folds of the average or median value of the set (β>0). In contrast, when the determination index 809 shows dissimilarity, the determination threshold may be, for example, defined to be a times as large as the maximum value of the set (α>1) or may be defined to be β times as large as the average or median value of the set (β>0). On the basis of the determination index set, the determination threshold may be defined to any value which can be selected for every target category depending on the species of an organism provided as an examination target, the type of an analysis method using pattern recognition, the determination accuracy of interest, and so on.

As a method of confirming whether the determination threshold as defined above is appropriately defined or not, there is a method where a sample which has been previously revealed to be not included in the selected target category is used as an unknown sample 105. By carrying out the processing for determining the biospecies of the unknown sample, which has been described above with reference to FIG. 1, an examination for determining whether the unknown sample leads to be “indeterminable” in result can be carried out to confirm whether the determination threshold is correctly defined or not.

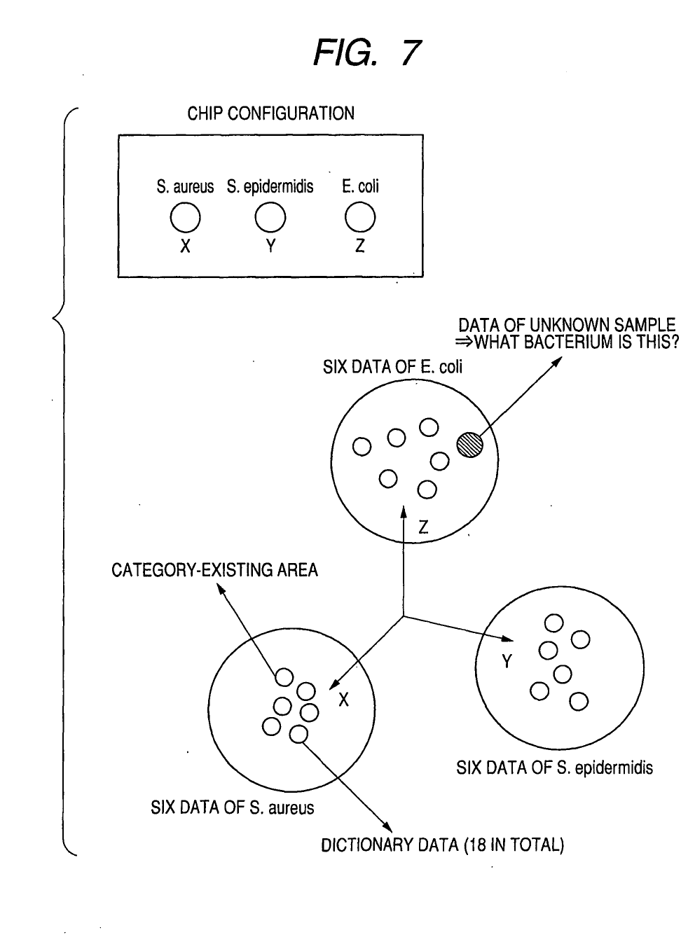

Next, the method (2), the method of defining a determination threshold, will be described below. In FIG. 10, another example for defining the determination threshold is illustrated. The following description will describe a method of defining a determination threshold when the k-Nearest-Neighbor (specifically, k=1) is selected as a pattern recognition algorithm and the Euclid norm is employed as a norm. In this case, if the determination index calculated from the image data obtained by analyzing the unknown sample shows dissimilarity, the result of “indeterminable” can be obtained when the determination index is larger than the defined determination threshold. For defining the determination threshold, one target category S1 is chosen first and then all of known samples belonging to S1 are hybridized and image data obtained is then stored in an external storage means. Combinations of two arbitrary image data belonging to S1 are selected from the whole image data stored in the storage means and a Euclid distance between vector data is then calculated, where the vector data are composed of fluorescence intensities disposed on the locations of probes recognized on the basis of the two image data described above. Next, a combination of two species data not selected in the foregoing is newly selected, and a Euclid distance is calculated on the basis of the two data newly selected in the same manner as that described above. Therefore, as described above, such procedures allow the Euclid distance to be calculated on the basis of each of the combinations with respect to the image data assembly belonging to S1 and stored in the external storage means. FIG. 7 represents a case in which six known samples belonging to the target category are prepared. In this case, the number of the Euclid distances calculated on the basis of two image data is ₆C₂=15.

FIG. 10 is a histogram in which the Euclid distances calculated on the basis of combinations of all image data belonging to the target category S1 are represented such that the X axis indicates the determination index. In FIG. 10, there are two crests in distribution of distances. It means that sample vectors belonging to the category are located in two regions. Therefore, from the histogram, the properties of the target category S1 can be confirmed, so that a method of defining an appropriate determination threshold can be chosen for every target category.

For instance, a representative statistical value, such as an average or median value, of the distance set can be employed as a determination threshold.

Next, for the determination threshold thus obtained, the processing for determining a biospecies of the unknown sample as described above with reference to FIG. 1 is carried out by the same way as that of the method (1) to confirm whether the determination threshold thus defined is appropriately defined. A sample previously proved to be not included in the selected target category is used as an unknown sample 105. The unknown sample is examined whether it is resulted in “indeterminable” to confirm that the determination threshold is correctly defined.

Next, a computer system as an information processing apparatus, programs, and each of processes such as an analysis method using image recognition, which can be used for the above method of determining a biospecies, will be described.

The above-mentioned determination of a biospecies can be automated by processing on a computer in accordance with a program created in advance. According to the present invention, an information processing apparatus for determining a corresponding biospecies by analyzing a sample in which a substance derived from an organism is supposed to be included includes:

a known sample image data inputting means for inputting image data specific to the biospecies obtained by analyzing known samples from a plurality of individuals whose corresponding biospecies are already revealed;

an unknown sample image data inputting means for inputting image data obtained by analyzing the unknown sample in a similar manner to a case of the known sample;

a first storage means for storing the image data captured;

means for defining a determination threshold with respect to a biospecies corresponding to the known sample on the basis of the plurality of analysis data obtained from the known samples;

a biospecies determining means for deciding whether the determination is possible or not on the basis of the determination threshold with respect to image data from the unknown sample, and determining the biospecies corresponding to the unknown sample when the determination is decided as possible;

a second storage means for storing a determination result obtained by the biospecies determining means; and

an output means for outputting the determination result stored in the second storage means.

It is preferable that:

the number of the individuals is equal to or greater than three;

the image data from the individuals are stored in the first storage means; and

the determination threshold is defined on the basis of a program including the steps of:

(a) carrying out a process on each of the image data and obtaining a determination index set composed of three or more determination indices, the process including selecting and removing image data on one individual from image data on the three or more individuals, creating an identification dictionary using image data on the remaining plurality of individuals, and obtaining a determination index by determining the image data previously removed on the basis of the obtained identification dictionary; and

(b) defining the determination threshold from the determination index set.

It is also preferable that:

the number of the individuals is equal to or greater than three;

the image data from the individuals are stored in the first storage means; and

the determination threshold is defined on the basis of a program having the steps of:

(A) obtaining a distance set by obtaining a distance between image data on two individuals with respect to every combination of image data on two arbitrary individuals selected from image data on the three or more individuals; and

(B) defining the determination threshold from the distance set.

Further, according to the present invention, a program for determination of a biospecies, for causing a computer to execute determination of a biospecies corresponding to an unknown sample, comprising the steps of:

(1) calling a plurality of known sample image data from a first storage means that stores a plurality of image data corresponding to image data specific to a biospecies to be supposed as a result of determination with respect to the unknown sample obtained by analyzing known samples from a plurality of different individuals belonging to the biospecies to be supposed;

(2) reading out the unknown sample image data from the first storage means for storing a plurality of image data corresponding to image data obtained by analyzing the unknown sample in a similar manner to a case of the known sample;

(3) defining a determination threshold by selecting one of the unknown sample image data and utilizing a relationship between the selected one and the remaining image data;

(4) determining a species of an organism corresponding to the unknown sample by processing the unknown sample image data on the basis of the determination threshold;

(5) storing a determination result obtained in the determination step (4) into a second storage means; and

(6) outputting the determination result stored in the second storage means.

It is preferable that:

the number of the individuals is equal to or greater than three;

the image data from the individuals are stored in the first storage means; and

the step (4) of determining a species includes the steps of:

(a) carrying out a process on each of the image data and obtaining a determination index set composed of three or more determination indices, the process including selecting and removing image data on one individual from image data on the three or more individuals, creating an identification dictionary using image data on the remaining plurality of individuals, and obtaining a determination index by determining the image data previously removed on the basis of the obtained identification dictionary; and

(b) defining the determination threshold from the determination index set.

It is also preferable that:

the number of the individuals is equal to or greater than three;

the image data from the individuals are stored in the first storage means; and

the step (4) of determining a species includes the steps of:

(A) obtaining a distance set by obtaining a distance between image data on two individuals with respect to every combination of image data on two arbitrary individuals selected from image data on the three or more individuals; and

(B) defining the determination threshold from the distance set.

Determination thresholds respectively for various known biospecies are stored in a storage means in advance. Depending on the kind of an unknown sample, a program is added with a step of selecting a required number of categories supposed to allow a substance derived from an organism in the unknown sample to show its presence. As a result, the number of categories to be investigated whether each of them is indeterminable can be effectively reduced, so that an efficient process for determination becomes possible.

By the way, the above program may be retained in the storage means of a computer system or may be stored in a recording medium and then distributed to the user. Alternatively, the program may be distributed through a network system.

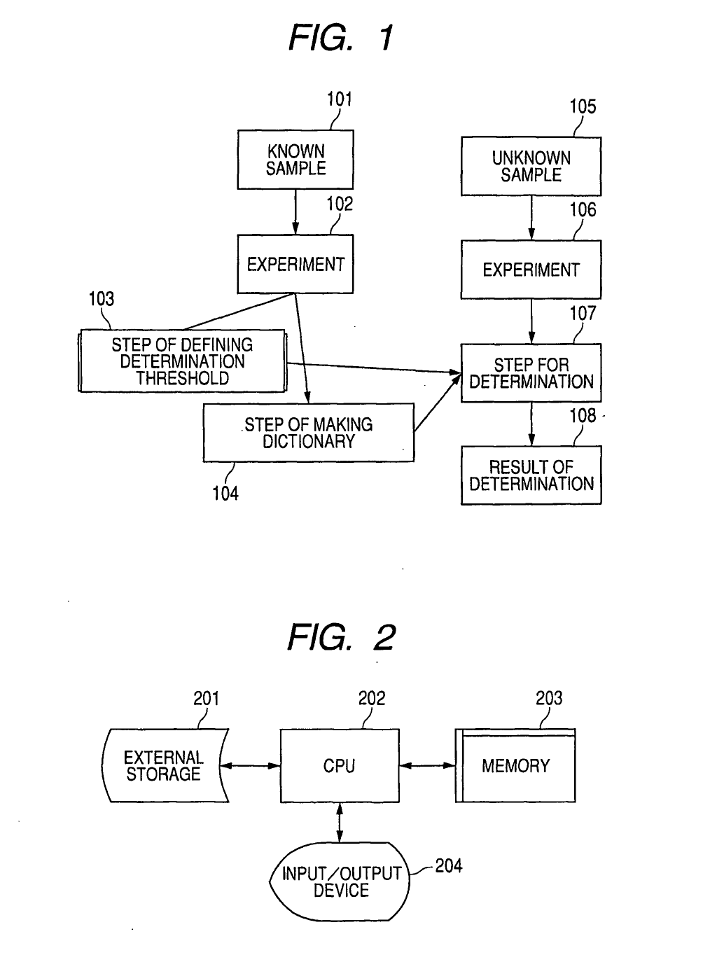

FIG. 2 is a block diagram showing an example of the configuration of an information processing apparatus using a computer system capable of carrying out the method of determining a biospecies. The apparatus is constructed of at least an external storage device 201, a central-processing unit (CPU) 202, a memory 203, and an input/output (I/O) device 204. The external storage device 201 retains a program configured as described above to carry out the determination of a biospecies, as well as image data as a result of analysis utilizing hybridization reactions with known and unknown samples. The external storage device 201 is allowed to further retain the results of determination using a determination threshold. The central-processing unit (CPU) 202 executes the program for determining a biospecies and controls all of the devices. The memory 203 is responsible for temporary storing the program used by the central-processing unit (CPU) 202 and also temporary storing a subroutine and data. The I/O device 204 carries out an interaction with the user. In many cases, the user can trigger the program execution through the I/O device. In addition, the user can see the results and control the program's parameters through the I/O device.

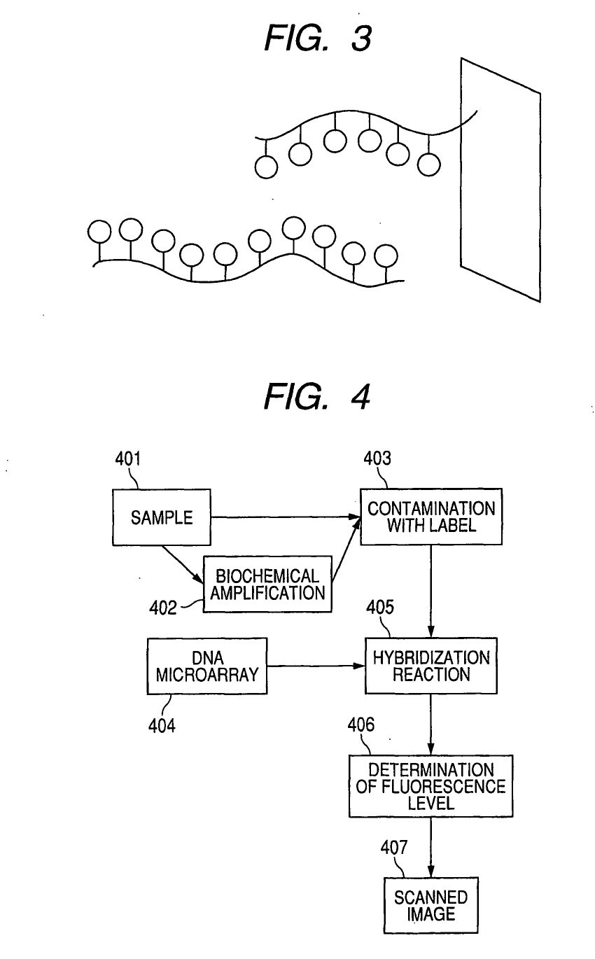

FIG. 3 is a diagram that illustrates an event of hybridization on a DNA microarray. In most cases within an organism, DNA exists in a double helix structure and the coupling between two strands is realized by a hydrogen bond between bases. In contrast, RNA often exists in a single strand. The bases include four different types A, T, G, and C for DNA and four different types A, U, G, and C for RNA, respectively. The base pairs capable of forming their respective hydrogen bonding are the pair of A and T(U) and the pair of G and C. In general, the term “hybridization reaction” refers to the state in which single-stranded nucleic acid molecules are partially coupled together through their partial base sequences in the molecules. In the example shown in FIG. 3, a nucleic acid molecule (probe) attached on a substrate on the upper side of the figure is shorter than a nucleic acid molecule in a sample on the lower side of the figure. When the nucleic acid molecule in the sample contains the base sequence of the probe, the hybridization reaction can complete well and the nucleic acid molecule in the sample can be trapped in a DNA microarray.

Next, referring now to FIG. 4, the whole experimental procedure for obtaining image data using a DNA microarray will be described. A “sample” 401 is a substance derived from an organism of interest, for example a liquid or an individual containing or supposed to contain nucleic acid (including one being retained in cells). Microorganisms including bacteria of, for example, tissues taken from humans, animals, and so on, and all Of materials supposed to contain substances derived therefrom may each be provided as a source of the unknown sample 401. For instance, when the present invention is applied for specifying a bacterial species causing an infection disease, the source may be any of body fluids such as blood, expectorated sputum, gastric juice, vaginal secretion, and oral mucosal fluid and excretion products such as urea and feces from humans and animals such as domestic animals. In addition, media potentially causing contamination with bacteria, including food products potentially causing food-poisoning or contamination, drinking water, and environmental water such as hot-spring water, may be used as sources of unknown samples. Furthermore, animals and plants subjected to, for example, quarantine for import and export procedures may be used as test substances. In the case of known samples, those prepared from known species of microorganisms may be appropriate.

Next, if required, the nucleic acid provided as the sample 401 is amplified using a method for “biochemical amplification” of 402. For instance, when the present invention is applied for specifying a bacterial species causing an infection disease, the target nucleic acid may be amplified using a PCR method with a PCR-reaction primer designed for the detection of 16s rRNA, and, for example, an additional PCR reaction using a PCR-amplified product may be carried out to adjust the amplification. In addition, the amplification may be adjusted using another amplification, method such as an LAMP method instead of the PCR.

Subsequently, the amplified sample or the sample 401 itself is labeled by any of various labeling methods for visualization. The labeling substance generally used is a fluorescent substance such as Cy3, Cy5, or Rhodamine. In addition, in the experimental procedure of biological amplification of 402, a labeling molecule may be mixed.

Furthermore, the nucleic acid added with the labeling molecule is subjected to a hybridization reaction (405) with a DNA microarray 404 shown in FIG. 4. This event may proceed as shown in FIG. 3. For example, in the case of applying the present invention to specify a bacterial species causing an infection disease, the DNA array 404 becomes one where a probe specific to the bacterial species is immobilized on a substrate. Probes for the respective bacteria have specificity against the bacteria much higher than, for example, a genome portion that encodes 16s rRNA and are designed to promise sufficient hybridization sensitivities without causing variations in the respective probe base sequences “as much as possible”. A carrier (substrate) for immobilizing probes of the DNA array 404 may be a flat substrate such as a glass substrate, a plastic substrate, or a silicon wafer. In addition, no influence on the embodiments and advantages of the present invention will occur even if a three-dimensional structure having an uneven surface, a spherical structure such as a bead, as well as a stick-like, corded, or filamentous structure is used.

In general, the surface of the substrate used may be processed so that the probe DNA can be immobilized thereon. In particular, a substrate having the surface on which a functional group is introduced so that a chemical reaction can be allowed is a preferable configuration with respect to reproducibility because the binding of probe is being stable during the hybridization reaction. A method of immobilizing a probe may be, for example, one in which a combination of a maleimide group and a thiol (—SH) group is employed to immobilize the probe on a substrate. In other words, the thiol (—SH) group is coupled with the terminal of a nucleic acid probe and then processed so that the surface of a solid phase has the maleimide group. As a result, the thiol group of the nucleic acid probe supplied to the surface of the solid phase and the maleimide group on the surface of the solid phase react to immobilize the probe. As a method for introduction of a maleimide group, an aminosilane coupling agent is subjected to a reaction with a glass substrate at first and an amino group thereof is then subjected to a reaction with an EMCS agent (N-(6-Maleimidocaproyloxy)succinimide: manufactured by DOJINDO Laboratories) to introduce the maleimide group. The introduction of the SH group into DNA can be carried out using 5′-Thiol-Modifier C6 (manufactured by Glen Research, Co., Ltd.) on a DNA automatic synthesizer. The combinations of functional groups to be used for immobilization include a combination of an epoxy group (on the solid phase) and an amino group (on the terminal of the nucleic acid probe) in addition to the combination of the thiol group and the maleimide group described above. In addition, the surface processing with any of various silane coupling agents may be also effective, so that oligonucleotide having a functional group introduced therein capable of reacting with a functional group introduced by the silane coupling agent can be used. Furthermore, a method of coating a resin having a functional group may be also utilizable.

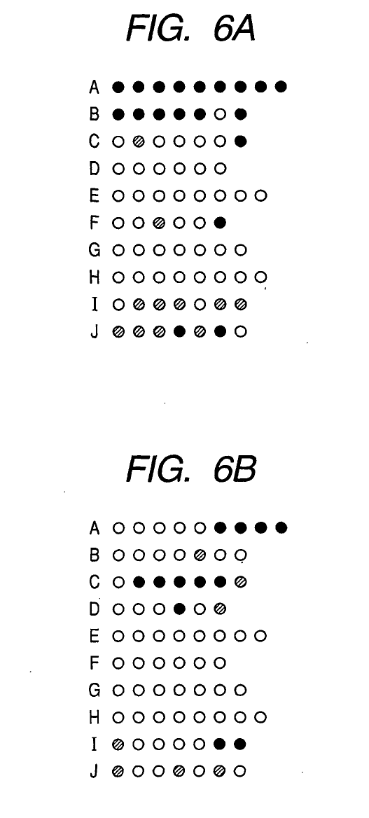

After the hybridization reaction has been carried out, the surface of the DNA microarray 404 is washed and a nucleic acid unattached with the probe is then removed, followed by drying in general and measuring an amount of fluorescence 405. Subsequently, excitation light is applied to the substrate of the DNA microarray to obtain an image on which the fluorescence intensity thereof is measured (406). The image (406) is then provided as image data. Examples of the image data are shown in FIGS. 6A and 6B, respectively. Different image data (images) are obtained in FIGS. 6A and 6B in correspondence with different known samples.

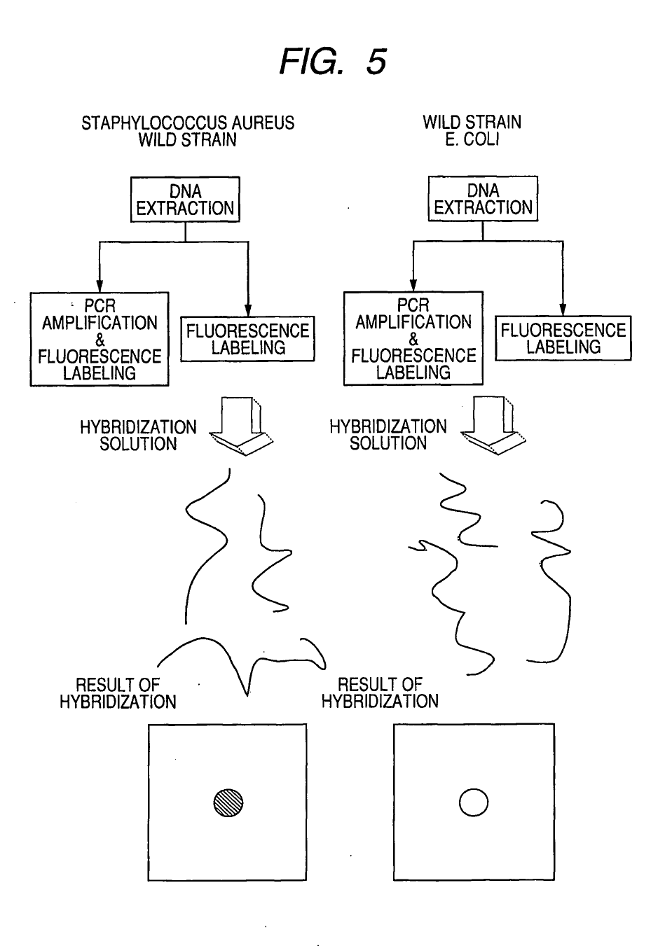

Next, referring now to FIG. 5, the principle of the DNA microarray for specifying a bacterial species causing an infection disease will be described. The DNA microarray shown in FIG. 5 is made for the purpose of specifying, for example, Staphylococcus aureus. The left line is a processed line derived from the wild strain of Staphylococcus aureus, while the right line is a processed line derived from the wild strain of Escherichia coli. For instance, the left may be considered to be a flow for processing the blood of a patient infected with Staphylococcus aureus, while the right may be considered to be a flow for processing the blood of a patient infected with Escherichia coli.

Both cases are basically subjected to the same processing. In other words, for example, DNA is initially extracted from the blood, sputum, or the like of the patient infected with the bacterial species. On this occasion, in general, human DNA from somatic cells of the patient may be included.

If the amount of the extracted DNA is small, the extracted DNA can be amplified using a PCR method or the like. On this occasion, in general, the extracted DNA may be mixed with a fluorescent substance or a substance capable of coupling with the fluorescent substance as a label. If the extracted DNA is not amplified, the extracted DNA is used and mixed with a fluorescent substance or a substance capable of coupling with the fluorescent substance as a label while a complementary strand is made. Alternatively, the directly extracted DNA is added with a fluorescent substance or a substance capable of coupling with the fluorescent substance as a label.

In general, for carrying out PCR amplification, portion of a base sequence that constitutes ribosomal RNA, the so-called 16s rRNA, is amplified for the purpose of specifying a bacterial species causing an infection disease. In this case, for the PCR primer of Staphylococcus aureus on the left and the PCR primer of Escherichia coli on the right, almost the same one can be used. More specifically, a primer set capable of amplifying any portion that encodes any bacterial 16s rRNA is employed to carry out multiplex PCR.

If a DNA microarray designed for the purpose of determining Staphylococcus aureus functions correctly, spots are positively reacted in a hybridization solution on the left but spots are negatively reacted in a hybridization solution on the right. Likewise, if a DNA microarray for the determination of Escherichia coli functions correctly, spots are negatively reacted in a hybridization solution on the left but spots are positively reacted in a hybridization solution on the right.

The fluorescence intensities from the positively reacted spots are measured and then subjected to a scan-image processing shown in FIG. 4, thereby obtaining image data. Here, when samples from different individuals belonging to the same species are used under the same analysis conditions to obtain their respective image data and the same fluorescence intensity therefrom is constantly obtained, the fluorescence intensity may be used as a dictionary. In actual, however, variations in fluorescence intensity occur. Thus, in some cases, it is difficult to obtain a clear norm to make a judgment as to whether image data from an unknown sample is in the range of such variations or deviates from the range to determine that the data is not included in a known category. Furthermore, as indicated in examples described later, cross hybridization may occur depending on the kind of probe. In the present invention, therefore, the creation of an identification dictionary and the definition of a determination threshold using samples from many different individuals belonging to the same species as shown in FIG. 8 make a clear norm as to whether the determination of an unknown sample is carried out for each of the categories.

Hereinafter, a concrete example of a method of acquiring analysis data that can be used in the method of determining a biospecies of the present invention will be provided. By the way, the present invention may be used for not only the specification of bacterial species causing an infection disease which will be described below but also the determination of the constitution of a human, such as MHC, and the analysis of DNA or RNA related to diseases such as cancer.

### Example 1

### <Preparation of Probe DNA>

A nucleic acid sequence (I-n) (n is a numeral) represented below was designed as a probe for detecting the bacterial species, Enterobacter cloacae. To be specific, probe base sequences represented below were chosen from a genome portion encoding 16s rRNA. Those probe base sequences have extremely high specificities against the bacterial species and are deigned to promise sufficient hybridization sensitivities without causing variations in the respective probe base sequences “as much as possible”.

I-1: CAgAgAgCTTgCTCTCgggTgA
I-2: gggAggAAggTgTTgTggTTAATAAC
I-3: ggTgTTgTggTTAATAACCACAgCAA
I-4: gCggTCTgTCAAgTCggATgTg
I-5: ATTCgAAACTggCAggCTAgAgTCT
I-6: TAACCACAgCAATtgACgTTACCCg
I-7: gCAATTgACgTTACCCgCAgAAgA

The above probe was allowed to introduce a thiol group, which was provided as a functional group for immobilization on a DNA microarray, into the 5′ terminal of the nucleic acid thereof by a routine procedure after synthesis. After the introduction of the functional group, the probe was purified and freeze-dried. The freeze-dried probe for an internal standard was stored in a refrigerator at 30° C.

On the other hand, for Staphylococcus aureus (A-n), Staphylococcus epiderimidis (B-n), Escherichia coli (C-n), Klebsiella pneumoniae (D-n), Pseudomonas aeruginosa (E-n), Serratia marcescens (F-n), Streptococcus pneumoniae (G-n), Haemophilus influenzae (H-n), and Enterococcus faecalis (J-n) (n is a numeral), probe sets represented below were also prepared in the same manner as that described above.

A-1: gAACCgCATggTTCAAAAgTgAAAgA
A-2: CACTTATAgATggATCCgCgCTgC
A-3: TgCACATCTTgACggTACCTAATCAg
A-4: CCCCTTAgTgCTgCAgCTAACg
A-5: AATACAAAgggCAgCgAAACCgC
A-6: CCggTggAgTAACCTTTTAggAgCT
A-7: TAACCTTTTAggAgCTAgCCgTCgA
A-8: TTTAggAgCTAgCCgTCgAAggT
A-9: TAgCCgTCgAAggTgggACAAAT
B-1: gAACAgACgAggAgCTTgCTCC
B-2: TAgTgAAAgACggTTTTgCTgCACT
B-3: TAAgTAACTATgCACgTCTTgACggT
B-4: gACCCCTCTAgAgATAgAgTTTTCCC
B-5: AgTAACCATTTggAgCTAgCCgTC
B-6: gAgCTTgCTCCTCTgACgTTAgC
B-7: AgCCggTggAgTAACCATTTgg
C-1: CTCTTgCCATCggATgTgCCCA
C-2: ATACCTTTgCTCATTgACgTTACCCg
C-3: TTTgCTCATTgACgTTACCCgCAg
C-4: ACTggCAAgCTTgAgTCTCgTAgA
C-5: ATACAAAgAgAAgCgACCTCgCg
C-6: CggACCTCATAAAgTgCgTCgTAgT
C-7: gCggggAggAAgggAgTAAAgTTAAT
D-1: TAgCACAgAgAgCTTgCTCTCgg
D-2: TCATgCCATCAgATgTgCCCAgA
D-3: CggggAggAAggCgATAAggTTAAT
D-4: TTCgATTgACgTTACCCgCAgAAgA
D-5: ggTCTgTCAAgTCggATgTgAAATCC
D-6: gCAggCTAgAgTCTTgTAgAgggg
E-1: TgAgggAgAAAgTgggggATCTTC
E-2: TCAgATgAgCCTAggTCggATTAgC
E-3: gAgCTAgAgTACggTAgAgggTgg
E-4: gTACggTAgAgggTggTggAATTTC
E-5: gACCACCTggACTgATACTgACAC
E-6: TggCCTTgACATgCTgAgAACTTTC
E-7: TTAgTTACCAgCACCTCgggTgg
E-8: TAgTCTAACCgCAAgggggACg
F-1: TAgCACAgggAgCTTgCTCCCT
F-2: AggTggTgAgCTTAATACgCTCATC
F-3: TCATCAATTgACgTTACTCgCAgAAg
F-4: ACTgCATTTgAAACTggCAAgCTAgA
F-5: TTATCCTTTgTTgCAgCTTCggCC
F-6: ACTTTCAgCgAggAggAAggTgg
G-1: AgTAgAACgCTgAAggAggAgCTTg
G-2: CTTgCATCACTACCAgATggACCTg
G-3: TgAgAgTggAAAgTTCACACTgTgAC
G-4: gCTgTggCTTAACCATAgTAggCTTT
G-5: AAgCggCTCTCTggCTTgTAACT
G-6: TAgACCCTTTCCggggTTTAgTgC
G-7: gACggCAAgCTAATCTCTTAAAgCCA
H-1: gCTTgggAATCTggCTTATggAgg
H-2: TgCCATAggATgAgCCCAAgTgg
H-3: CTTgggAATgTACTgACgCTCATgTg
H-4: ggATTgggCTTAgAgCTTggTgC
H-5: TACAgAgggAAgCgAAgCTgCg
H-6: ggCgTTTACCACggTATgATTCATgA
H-7: AATgCCTACCAAgCCTgCgATCT
H-8: TATCggAAgATgAAAgTgCgggACT
J-1: TTCTTTCCTCCCgAgTgCTTgCA
J-2: AACACgTgggTAACCTACCCATCAg
J-3: ATggCATAAgAgTgAAAggCgCTT
J-4: gACCCgCggTgCATTAgCTAgT
J-5: ggACgTTAgTAACTgAACgTCCCCT
J-6: CTCAACCggggAgggTCATTgg
J-7: TTggAgggTTTCCgCCCTTCAg

### <Preparation of PCR Primer for Sample Amplification>

Nucleic acid sequences represented in Table 1 were designed as a PCR primer for the amplification of 16s rRNA nucleic acid (target nucleic acid) for detecting a prophlogistic bacillus. To be specific, a probe set for specific amplification of a genome portion encoding 16s rRNA, i.e., primers where both terminal portions of a 16s rRNA coding region of about 1,500 in base length having specific melting temperatures even up as far as possible, were designed. By the way, a plurality of different primers were designed such that a mutant strain or a plurality of 16s rRNA coding regions retained on the genome could be also simultaneously amplified.

TABLE 1

| | Primer | |
|---|---|---|
| | No. | Sequence |
| Forward Primer | F-1 | 5′GCGGCGTGCCTAATACATGCAAG3′ |
| | F-2 | 5′GCGGCAGGCCTAACACATGCAAG3′ |
| | F-3 | 5′GCGGCAGGCTTAACACATGCAAG3′ |
| Reverse Primer | R-1 | 5′ATCCAGCCGCACCTTCCGATAC3′ |
| | R-2 | 5′ATCCAACCGCAGGTTCCCCTAC3′ |
| | R-3 | 5′ATCCAGCCGCAGGTTCCCCTAC3′ |

The primers represented in the table were purified with high-performance liquid chromatography (HPLC) after synthesis and then mixed with three different forward primers and three different reverse primers, while being dissolved in a TE buffer so that each of the primers had a final concentration of 10 pmol/μl.

<Extraction of Enterobacter cloacae Genome DNA (Model Sample)>

### (Incubation of Microorganism and Pretreatment of Genome DNA Extraction)

First, the standard strain of Enterobacter cloacae was incubated by a routine procedure.

The culture fluid of microorganism was collected in an amount of 1.0 ml (OD600=0.7) into a microtube of 1.5 ml in volume and then centrifuged to recover bacterial cells (8,500 rpm, 5 min, 4° C.). After removal of a supernatant, the recovered bacterial cells were added with 300 μl of Enzyme Buffer (50 mM Tris-HCl: pH8.0, 25 mM EDTA), followed by resuspending with a mixer. The resuspended bacterial fluid was re-centrifuged to recover bacterial cells (8,500 rpm, 5 min., 4° C.). After removal of a supernatant, the recovered bacterial cells were added with the following enzyme solution and then resuspended with a mixer.

Lysozyme: 50 μl (20 mg/ml in Enzyme Buffer)

N-Acetylmuramidase SG.: 50 μl (0.2 mg/ml in Enzyme Buffer)

Next, the bacterial fluid resuspended by the addition of the enzyme solution was left standing in an incubator at 37° C. for 30 minutes to carry out cell-wall digestion.

### (Extraction of Genome)

The extraction of genome DNA from any of microorganisms described below was carried out using a nucleic acid purification kit (MagExtractor-Genome-: manufactured by TOYOBO). To be specific, first, 750 μl of a dissolution/adsorption solution and 40 μl of magnetic beads were added to the pretreated suspension of microorganism and then the whole was vigorously stirred for 10 minutes by using a tube mixer (Step 1). Secondly, a microtube was set on a separation stand (Magical Trapper) and then left standing for 30 seconds to accumulate the magnetic particles to the wall surface of the tube, followed by removal of a supernatant while the tube was kept on the stand (Step 2). Then, 900 μl of a cleaning solution was added and then resuspended by stirring for about 5 seconds with a mixer (Step 3). Subsequently, a microtube was set on a separation stand (Magical Trapper) and then left standing for 30 seconds to accumulate the magnetic particles to the wall surface of the tube, followed by removal of a supernatant while the tube was kept on the stand (Step 4). After Steps 3 and 4 had been repeated to carry out the second cleaning (Step 5), 900 μl of 70% ethanol was added and then resuspended by stirring for about 5 seconds with a mixer (Step 6). Next, a microtube was set on a separation stand (Magical Trapper) and then left standing for 30 seconds to accumulate the magnetic particles to the wall surface of the tube, followed by removal of a supernatant while the tube was kept on the stand (Step 7). After Steps 6 and 7 had been repeated to carry out the second cleaning with 70% ethanol (Step 8), 100 μl of pure water was added to the collected magnetic particles and then the whole was stirred for 10 minutes with a tube mixer.

Next, a microtube was set on a separation stand (Magical Trapper) and then left standing for 30 seconds to accumulate the magnetic particles to the wall surface of the tube, followed by the collection of a supernatant into a new tube while the tube was kept on the stand.

### (Examination of Collected Genome DNA)

The collected genome DNA of the microorganism (Enterobacter cloacae strain) was subjected to agarose electrophoresis and absorbance measurement at 260/280 nm by routine procedures to assay the quality (the amount of contaminant low-molecular nucleic acid, the degree of decomposition) of the genome DNA and the amount thereof recovered. In this example, about 10 μg of genome DNA was recovered. Neither degradation of genome DNA nor contamination with rRNA was observed. The recovered genome DNA was dissolved in a TE buffer to a final concentration of 50 ng/μl. The resulting product was used in the following steps.

### <Preparation of DNA Microarray>

### [1] Cleaning of Glass Substrate

A glass, substrate made of synthetic quartz (25 mm×75 mm×1 mm in dimensions, manufactured by IYAMA PRECISION GLASS) was placed in a heat-resistant, alkali-resistant rack and immersed in a cleaning solution for ultrasonic cleaning prepared at a predetermined concentration. After the substrate had been immersed in the cleaning solution overnight, ultrasonic cleaning was carried out for 20 minutes. Subsequently, the substrate was pulled out of the solution and lightly rinsed with pure water, followed by ultrasonic cleaning in ultrapure water for 20 minutes. Then, the substrate was immersed in an aqueous 1-N sodium hydroxide solution heated to 80° C. Subsequently, the substrate was washed with pure water and ultrapure water again, thereby preparing a quartz glass substrate for DNA chip.

### [2] Surface Treatment

A silane coupling agent KBM-603 (manufactured by Shin-Etsu Silicones) was dissolved in pure water to a concentration of 1% and then the solution was stirred at room temperature for 2 hours. Subsequently, the previously washed glass substrate was immersed in an aqueous solution of the silane coupling agent and left standing at room temperature for 20 minutes. The glass substrate was pulled out and the surface thereof was then lightly washed with pure water, followed by drying with nitrogen gas blown on both surfaces of the substrate. Next, the dried substrate was baked for 1 hour in an oven heated to 120° C. to complete the treatment with the coupling agent, thereby allowing the introduction of an amino group into the surface of the substrate. Then, N-(6-maleimidocaproyloxy)succinimide (hereinafter, abbreviated as EMCS) was dissolved in a mixture solvent of dimethyl sulfoxide and ethanol (1:1) to a final concentration of 0.3 mg/ml to prepare an EMCS solution. The baked glass substrate was left standing to cool and then immersed in the EMCS solution thus prepared at room temperature for 2 hours. This treatment allowed a reaction of an amino group introduced into the surface by the silane coupling agent with a succinimide group of EMCS, thereby introducing a maleimide group into the surface of the glass substrate. The glass substrate pulled out of the EMCS solution was washed with a mixture solvent in which the MCS described above was dissolved and then washed with ethanol, followed by drying under a nitrogen gas atmosphere.

### [3] Probe DNA

The previously prepared probe for the detection of a microorganism was dissolved in pure water and then the solution was divided so that each solution had a final concentration of 10 μM (at the time of dissolving ink), followed by freeze-drying to remove moisture contents.

### [4] DNA Ejection by BJ Printer and Binding to Substrate

An aqueous solution containing 7.5 wt % of glycerin, 7.5 wt % of thioglycol, 7.5 wt % of urea, and 1.0 wt % of Acetylenol EH (manufactured by Kawaken Fine Chemicals) was prepared. Then, each of seven different probes (Table 1) which had been previously prepared was dissolved in the mixture solvent described above to a normal concentration. An ink tank for a bubble-jet printer (trade name: BJF-850, manufactured by Canon) was filled with the resulting DNA solution, and was then attached to a printing head.

Here, the bubble-jet printer used herein is one modified so that it could print on a flat plate. In addition, the bubble jet printer is capable of spotting about 5 pl of the DNA solution with a pitch of 120 micrometers by inputting printing pattern in accordance with a predetermined method of creating a file. Subsequently, the modified bubble-jet printer was used to carry out a printing operation on a single glass substrate to form an array. After it had been confirmed that the printing had been completely carried out, the glass substrate was placed standing in a humidified chamber for 30 minutes to allow a maleimide group on the surface of the glass substrate to react with a thiol group of the terminal of a nucleic acid probe.

### [5] Cleaning

After the reaction for 30 minutes, the DNA solution remained on the surface was washed out by a 10-mM phosphate buffer (pH 7.0) containing 10.0 mM of NaCl, thereby obtaining a DNA microarray in which a single DNA strand was immobilized on the surface of the glass substrate.

### <Amplification and Labeling of Sample (PCR Amplification and Uptake of Fluorescent Label)>

The amplification of microorganism DNA to be provided as a sample and a labeling reaction will be described below.
Premix PCR reagent TAKARA ExTaq): 25 μl
Template Genome DNA: 2 μl (100 ng)
Forward Primer mix: 2 μl (20 pmol/tube each)
Reverse Primer mix: 2 μl (20 pmol/tube each)
Cy-3 dUTP (1 mM): 2 μl (2 nmol/tube)
H₂O: 17 μl
(Total: 50 μl)

A reaction solution having the above composition was subjected to an amplification reaction using a thermal cycler commercially available in the market in accordance with the following protocol.

(Step 1) 95° C., 10 min.

(Step 2) 92° C., 45 sec.

(Step 3) 55° C., 45 sec.

(Step 4) 72° C., 45 sec.

(Step 5) 72° C., 10 min.

(Steps 2 to 4 were Repeated 35 Times)

After the completion of the reaction; a purification column (QLAGEN QIAquick PCR Purification Kit) was used to remove the primer and an amplification product was quantified, resulting in a labeled sample.

### <Hybridization>

A detection reaction was carried out using the DNA microarray prepared in the section of <Preparation of DNA microarray> and the labeled sample prepared in the section of <Amplification and labeling of sample (PCR amplification and uptake of fluorescent label)>.

### (Blocking of DNA Microarray)

Bovine serum albumin (BSA Fraction V: manufactured by Sigma-Aldrich Japan) was dissolved in a 100-mM NaCl/10-mM phosphate buffer to a concentration of 1 wt %. Then, the DNA microarray prepared in the section of <Preparation of DNA microarray> was immersed in the solution for 2 hours at room temperature to carry out blocking on the DNA microarray. After the completion of the blocking, the DNA microarray was washed with a 2×SSC solution (300 mM of NaCl, 30 mM of sodium citrate (trisodium citrate dihydrate, C₆H₅Na₃.2H₂O) containing 0.1 wt % sodium dodecyl sulfate (SDS) and then rinsed with pure water followed by removal of water with a spin-dry device.

### (Hybridization)

The water-removed DNA microarray was set on a hybridization device (Hybridization Station: manufactured by Genomic Solutions Inc.) and a hybridization reaction was then carried out using the following hybridization solution under the following hybridization conditions.

### <Hybridization Solution>

6×SSPE/10% form amide/target (2nd PCR products, total volume)

(6×SSPE: 900 mM NaCl, 60 mM NaH₂PO₄.H₂O, 6 mM EDTA, pH 7.4)

### <Hybridization Conditions>

65° C., 3 min→92° C., 2 min→45° C., 3 hrs→Wash, 2×SSC/0.1% SDS, 25° C.→Wash, 2×SSC, 20° C.→(Rinse with H₂O: Manual)→Spin dry

### <Detection of Microorganism (Fluorescence Measurement)>

The DNA microarray after the completion of the hybridization reaction was subjected to fluorescence measurement using a fluorescence detector for DNA microarray (GenePix 4000B: manufactured by Axon Instruments).

Examples of images as the image data thus obtained are shown in FIGS. 6A and 6B, respectively. Here, in FIGS. 6A and 6B, the probe having stronger fluorescence intensity is represented by a denser color. FIG. 6A is an example of an image obtained when a sample containing the genome of Staphylococcus aureus was reacted with the DNA microarray, while FIG. 6B is an example of an image obtained when a sample containing the genome of Escherichia coli was reacted with the DNA microarray. Alphabetical characters written on the left side of the figure are those of the probe sequence. A to J represent probes designed to be specifically bound to Staphylococcus aureus (A), Staphylococcus epiderimidis (B), Escherichia coli (C), Klebsiella pneumoniae (D), Pseudomonas aeruginosa (E), Serratia marcescens (F), Streptococcus pneumoniae (G), Haemophilus influenzae (H), Enterobacter cloacae (I), and Enterococcus faecalis (J).

Ideally, only the probe on the row A of FIG. 6A showed higher fluorescence intensity and also only the probe on the row C of FIG. 6B showed higher fluorescence intensity. Ideal results of FIG. 6A are identical to the experimental results shown in FIG. 5.

However, as shown in FIGS. 6A and 6B, the actual results are below the ideal. That is, the so-called “cross hybridization reaction” occurs. In the case of FIG. 6A, some of probes on the rows other than the row A also showed higher fluorescence intensity. In the case of FIG. 6B, in addition, some of probes on the rows other than the row C also showed higher fluorescence intensity. In the case of FIG. 6B, furthermore, a probe having weak fluorescence intensity can be also found on the row C.

FIG. 7 illustrates this situation with the system of three probes. A DNA microarray having three different probes of S. aureus, S. epiderimidis, and E. coli were used and six different known samples were tested for these bacterial species. In general, if there are “N” probes, the experiment data can be N-dimensional vectors. In the case of FIGS. 6A and 6B, there are 72 probes in total, so that the experiment data can be 72-dimensional vectors. In the case of FIG. 7, there are three probes, so that the experiment data can be 3-dimensional vectors.

In the lower panels of FIG. 7, six different samples of each of three bacterial species (=18 data in total) were plotted on a three-dimensional coordinate system. As shown in the figure, if three probes are ideally very specific probes for three respective bacterial species, the vector data concentrate around the respective axes as shown in the lower panel of FIG. 7. However, there is data fluctuation, so that the data cannot concentrate on one point. In the case of the example shown in FIG. 7, the areas in which data exists for three different bacterial species are substantially different in size in descending order of E. coli, S. epiderimidis, and S. aureus.

Furthermore, a determination index set is derived for every bacterial species in accordance with the method of FIGS. 1 and 8, which are previously described, to define a determination threshold. Then, the defined determination threshold can be used for determining whether the determination of a bacterial species as a source of supplying an unknown sample is carried out or the determination thereof is not carried out.

### Example 2

The experimental data on DNA microarrays for Klebsiella pneumoniae and Serratia marcescens will be described below. Here, probes used are those of n=1 to 6, previously represented in S. aureus (A-n), S. epiderimidis (B-n), E. coli (C-n), K. pneumoniae (D-n), P. aeruginosa (E-n), S. marcescens (F-n), S. pneumoniae (G-n), H. influenzae (H-n), E. cloacae (I-n), and E. faecalis (J-n). Ultimately, the total number of probes is 10×6=60.

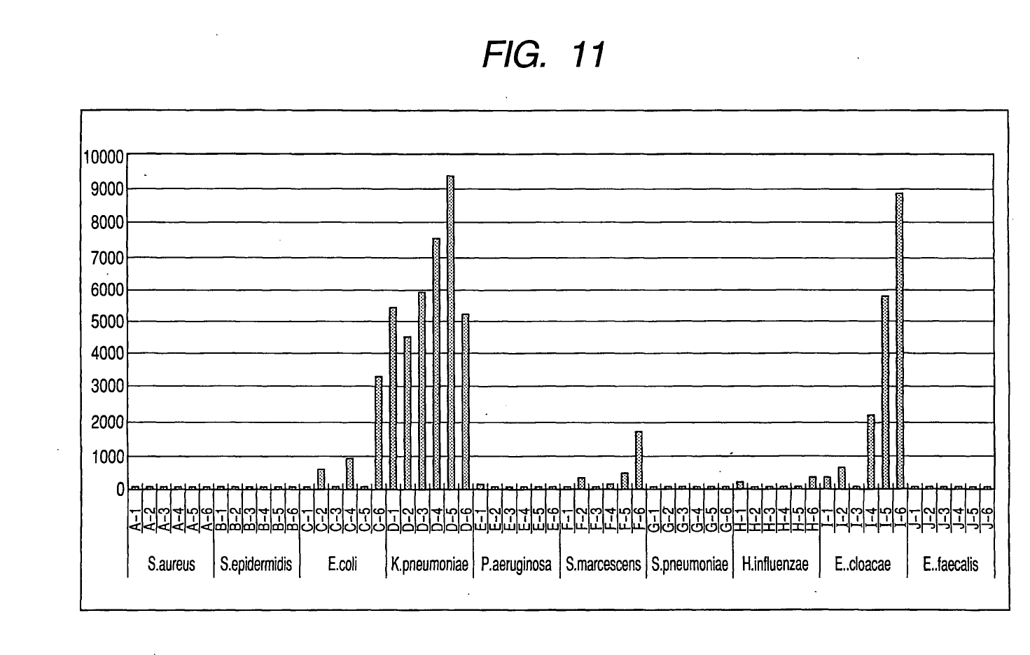

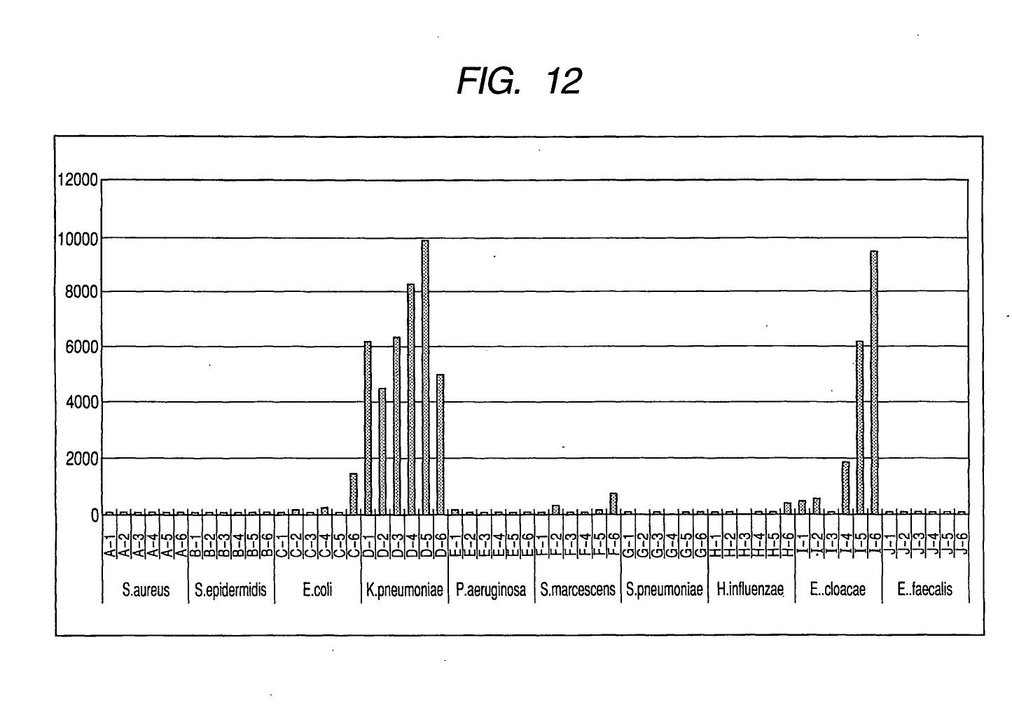

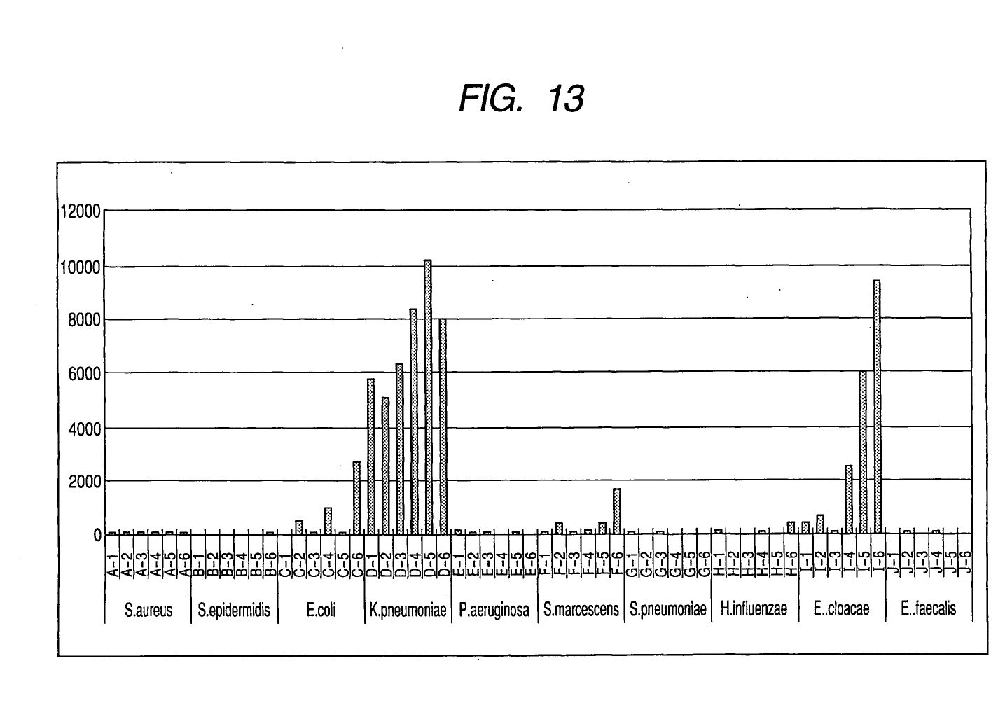

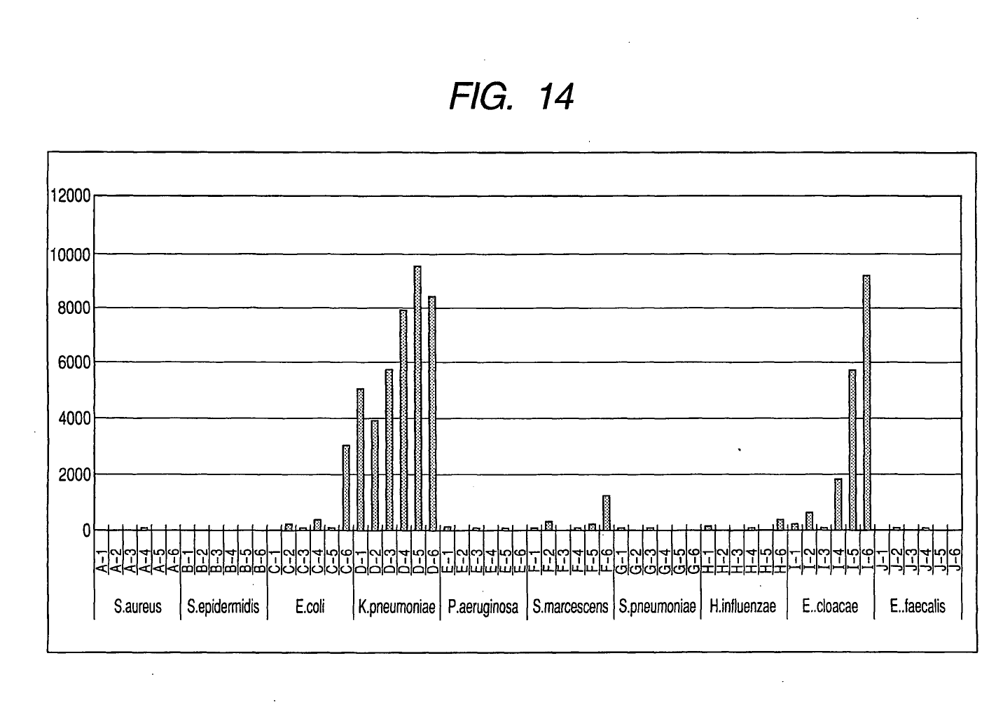

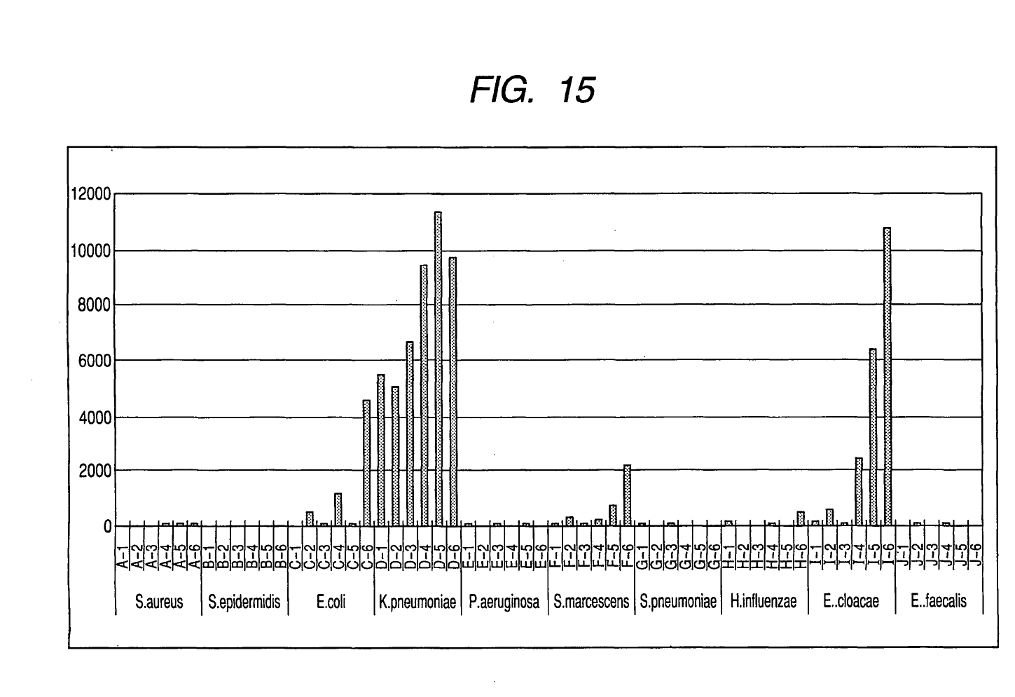

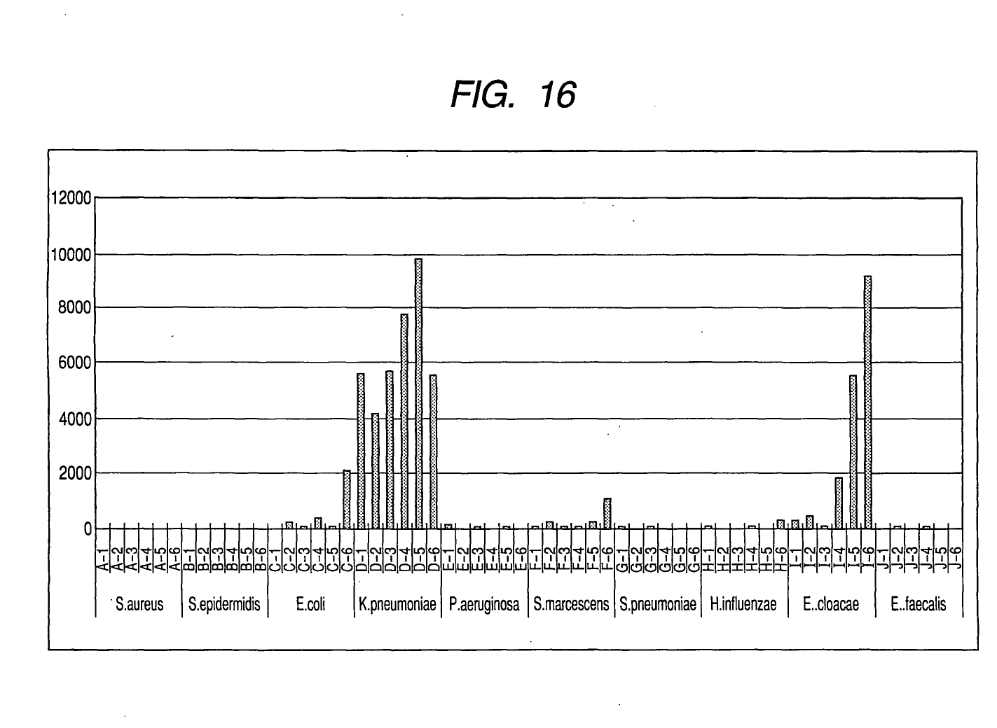

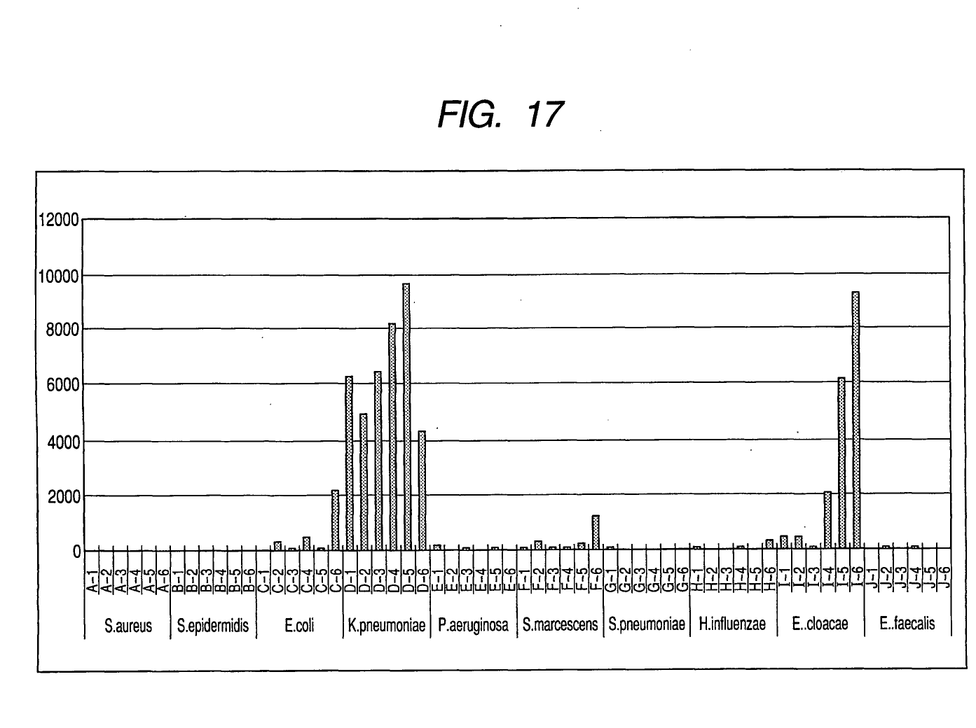

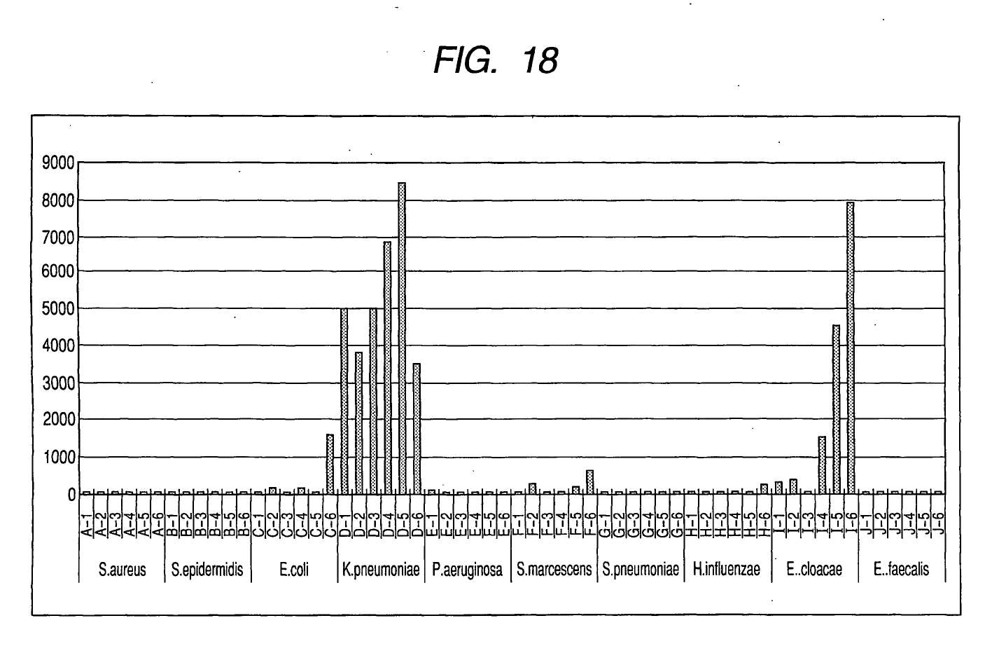

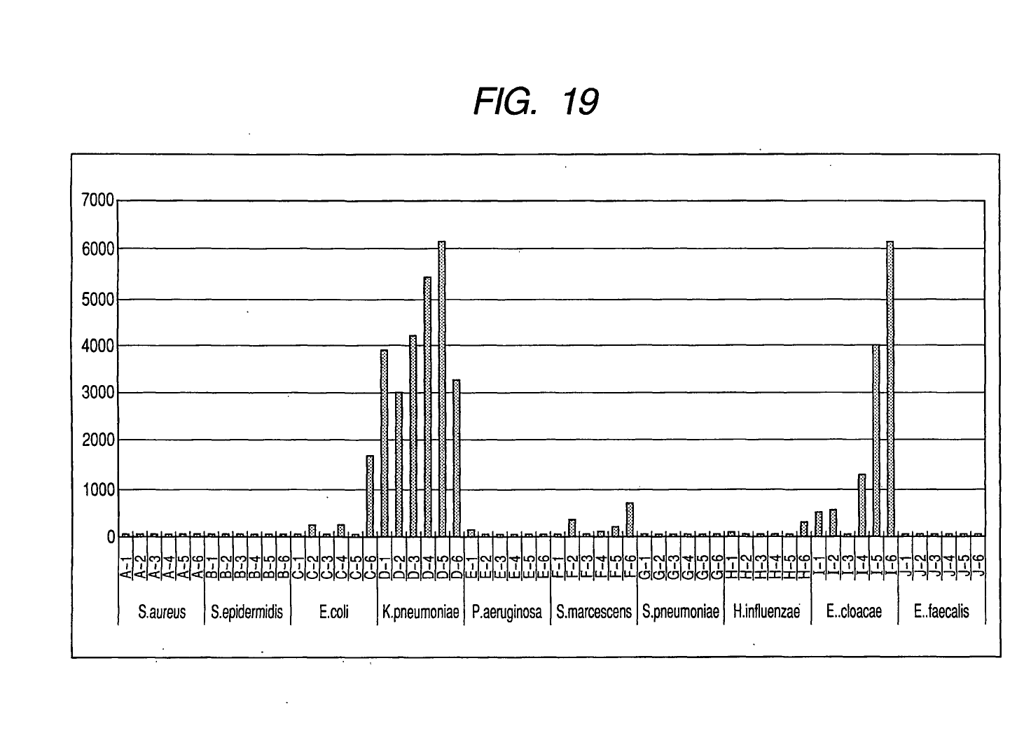

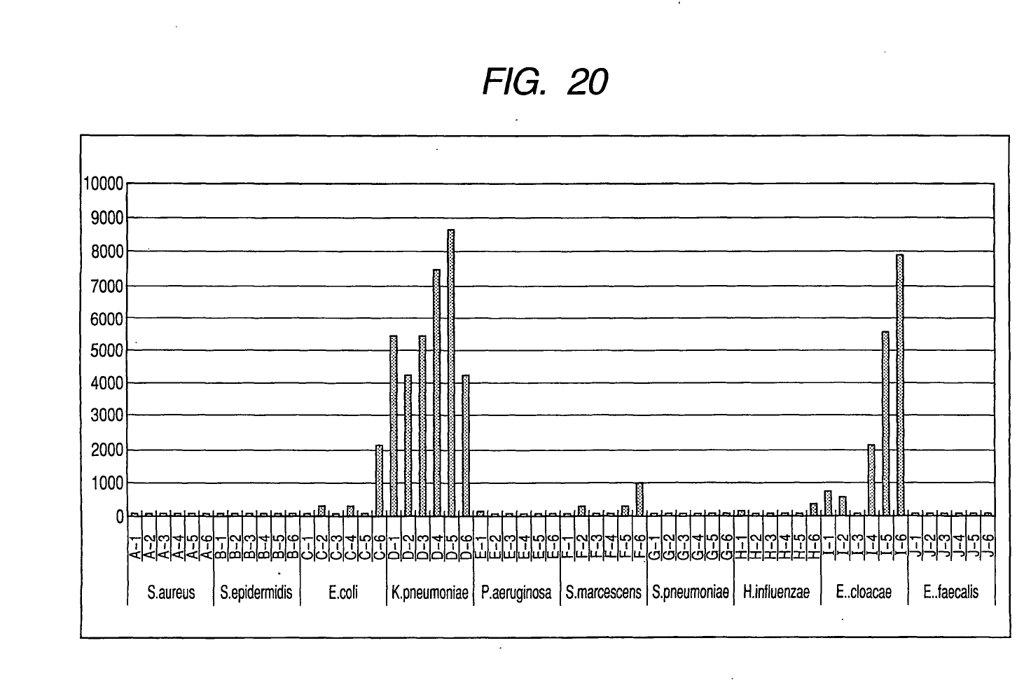

Experimental data on DNA microarrays for 10 different samples of K. pneumoniae are shown in FIGS. 11 to 20, respectively. In each figure, from the left to the right, the probes are arranged in the order of probe A-1, A-2, - - - , J-5, and J-6. As shown in the figures, the experimental data on the DNA microarray can be obtained as 60 values of fluorescence intensities, i.e., 60 dimensional vectors. First, for defining the distance between arbitrary vectors, normalization is conducted such that “each element of a vector is divided by the norm of the vector”. The equation can be described as follows:

$$( \begin{matrix}{y}_{1} \\ {y}_{2} \\ {y}_{3} \\ M \\ {y}_{60}\end{matrix} ) = ( \begin{matrix}{x}_{1} \\ {x}_{2} \\ {x}_{3} \\ M \\ {x}_{60}\end{matrix} ) \cdot \frac{1}{\sqrt{{\sum }_{k = 1}^{60} {( {x}_{k} )}^{2}}} \qquad [ Equation 1 ]$$

where a vector x is an original vector and a vector y is a vector after the normalization.

Therefore, the normalized vector has constantly a norm of 1. Here, the norm of the vector x (Euclid norm) in the “n” dimension can be defined by the following equation.

$$\sqrt{{\sum }_{k = 1}^{n} {( {x}_{k} )}^{2}} \qquad [ Equation 2 ]$$

Then, the distance between two vectors (vector a and vector b) after normalization can be defined by the following equation.

$${\sum }_{k = 1}^{60} {( {a}_{k} - {b}_{k} )}^{2} \qquad [ Equation 3 ]$$

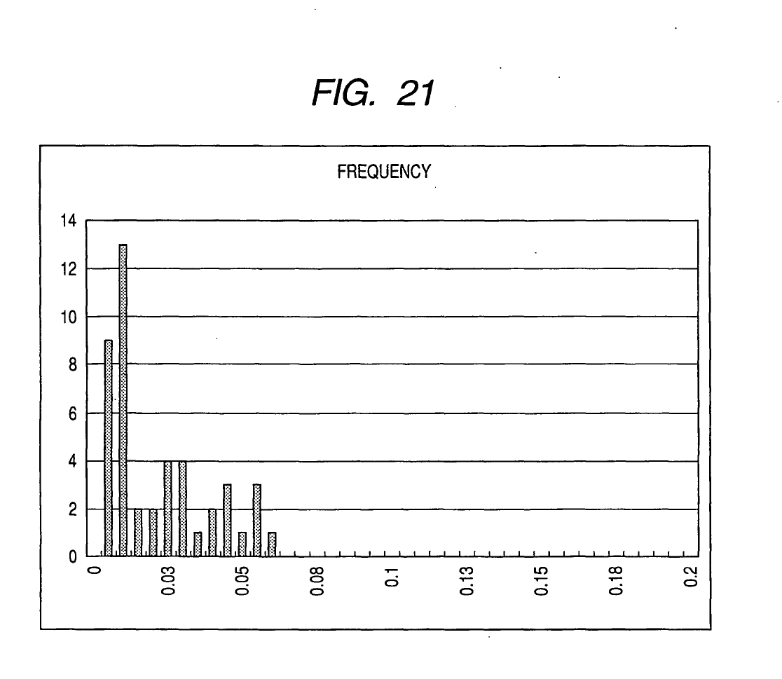

In this example, the distance definition of k-th nearest neighbor matching algorithm may be carried out as described above. The distance between an arbitrary pair from 10 samples is calculated and represented as a histogram on FIG. 21. The number of data reaches ₁₀C₂=45. From the figure, when the determination is carried out by applying the above k-th nearest neighbor algorithm to K. pneumoniae, one of the candidates of the determination threshold is the maximum value, 0.057. Alternatively, a 1.5- or 2-folded value with some allowance may be used.

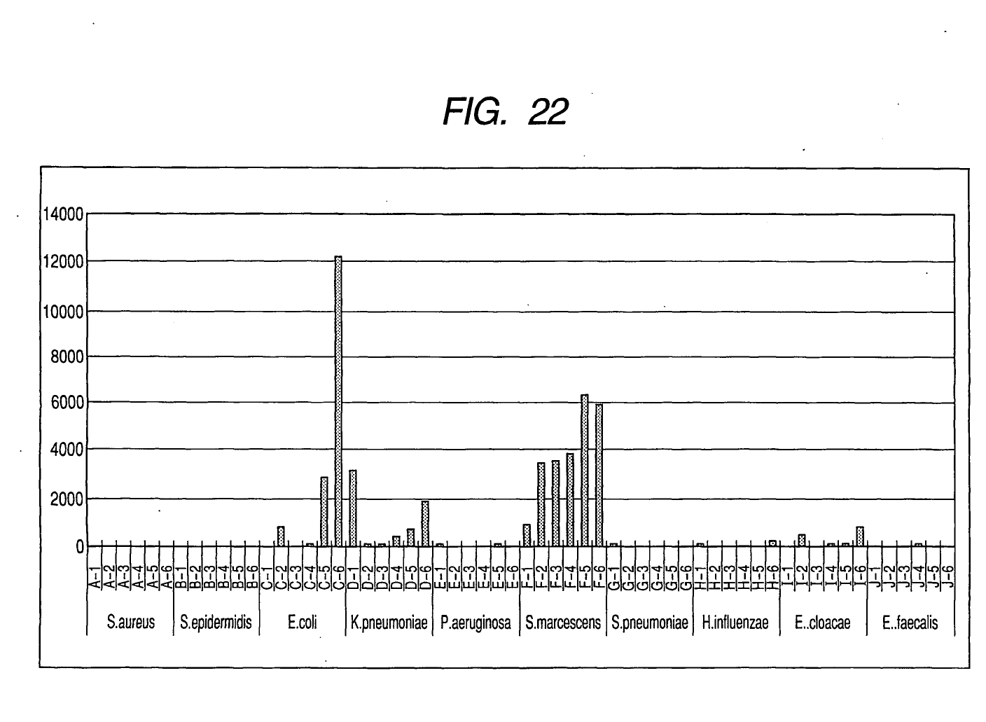

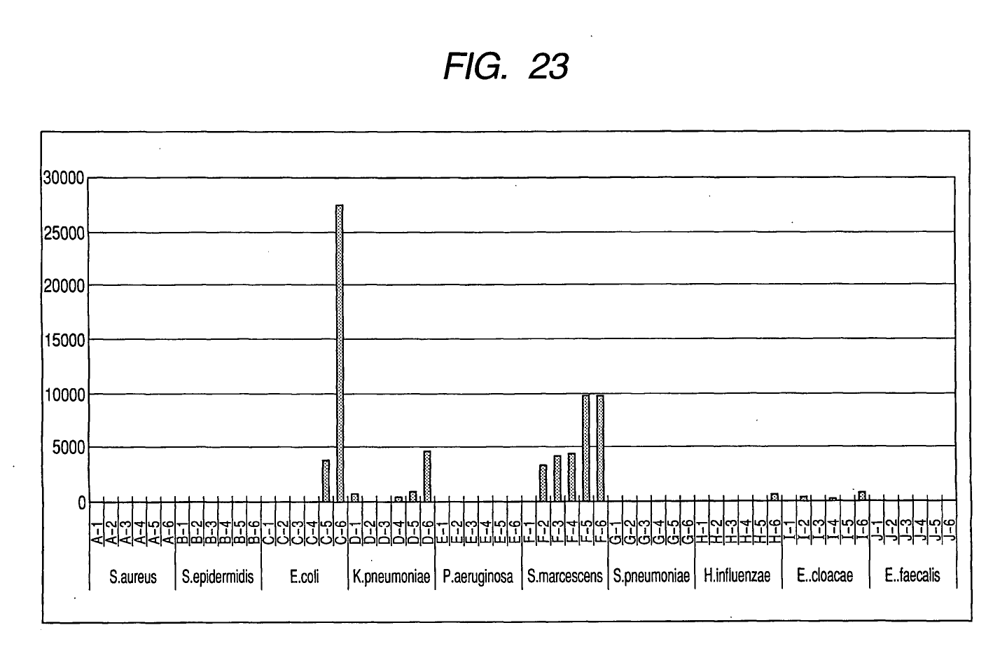

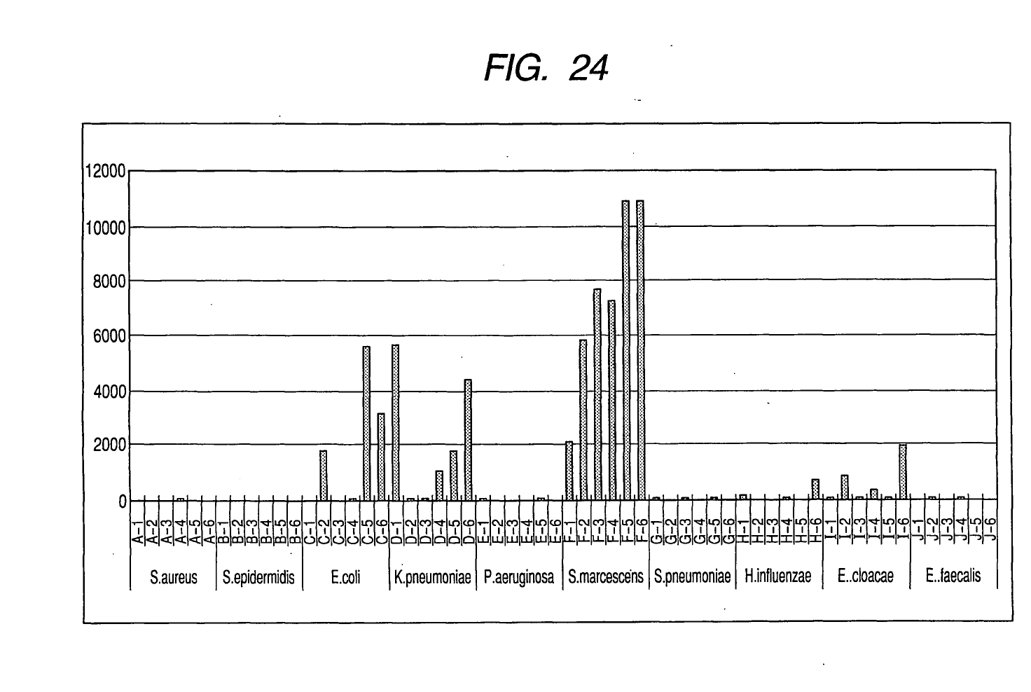

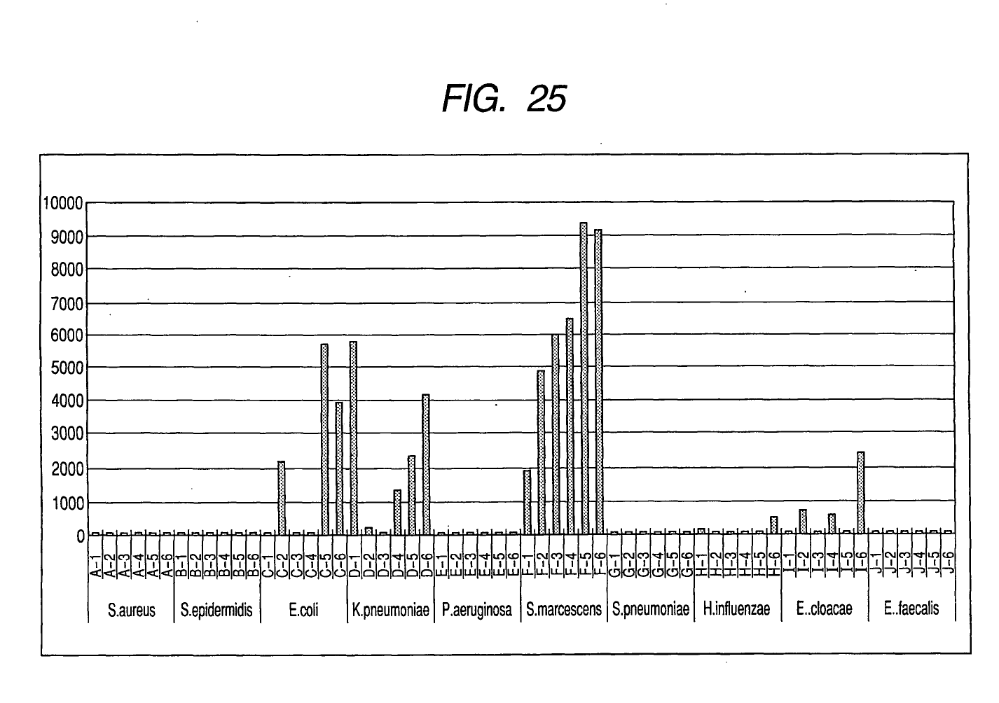

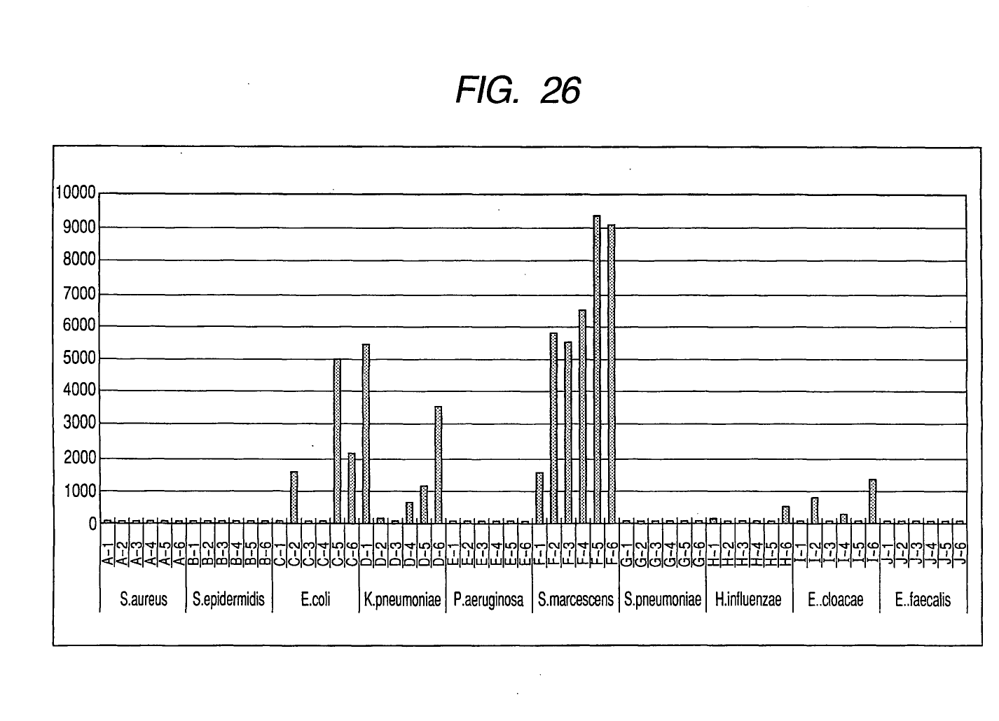

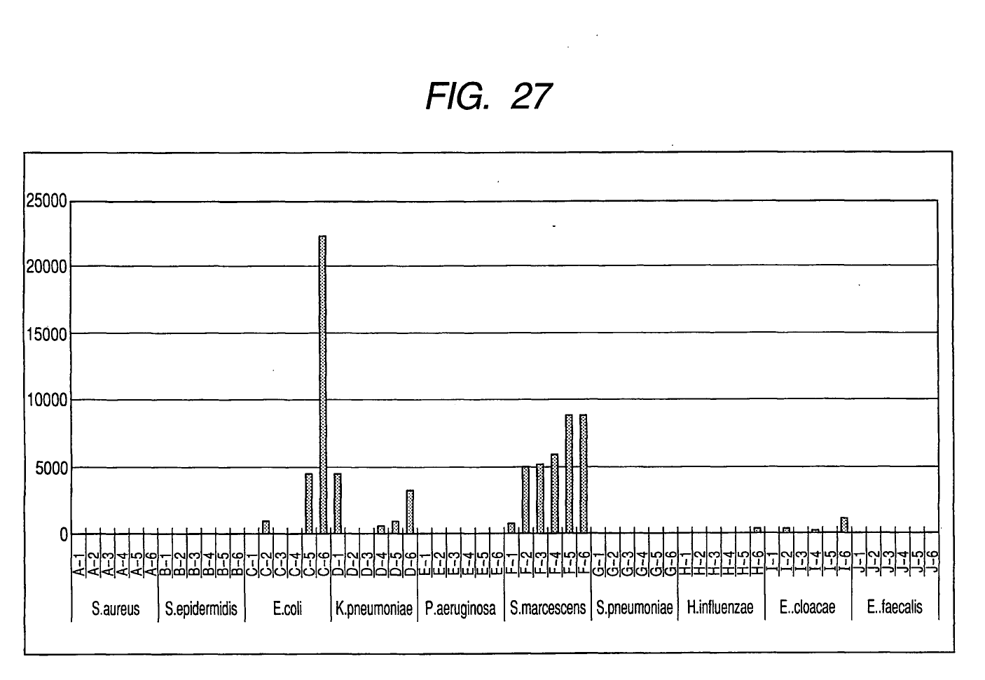

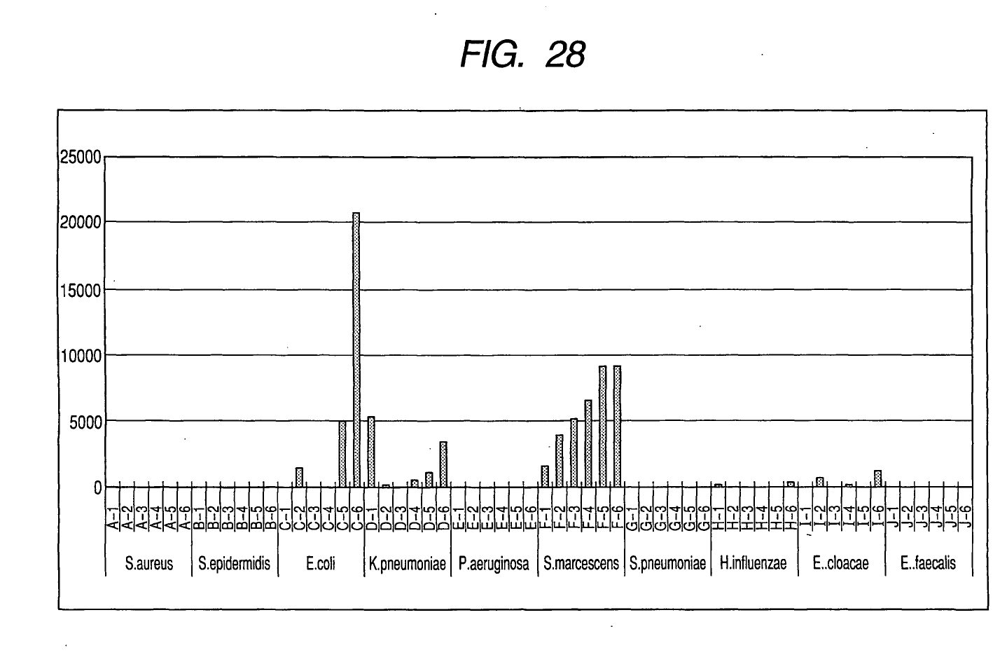

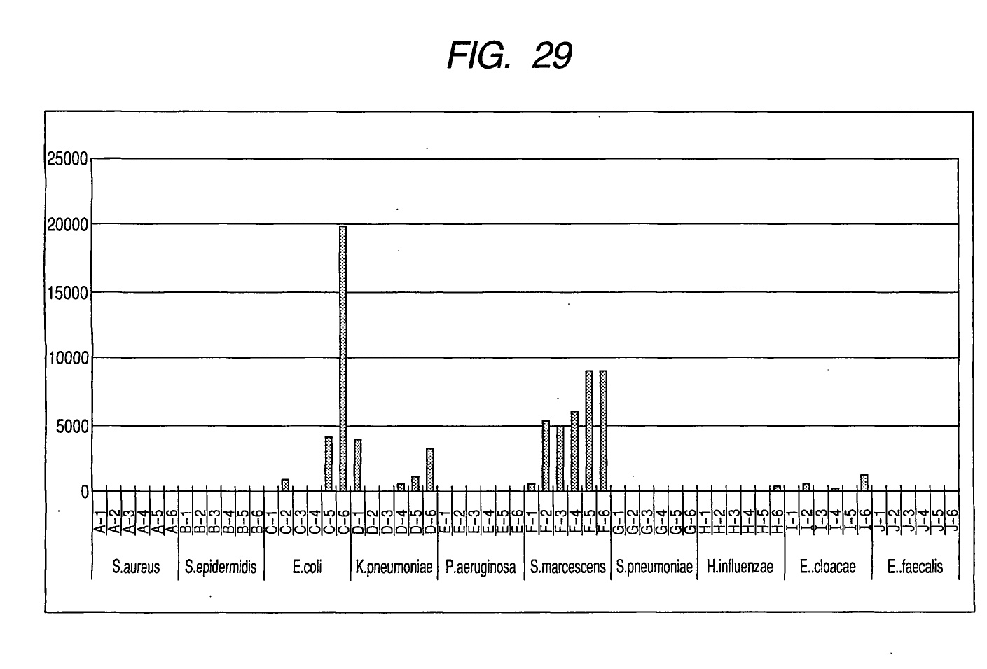

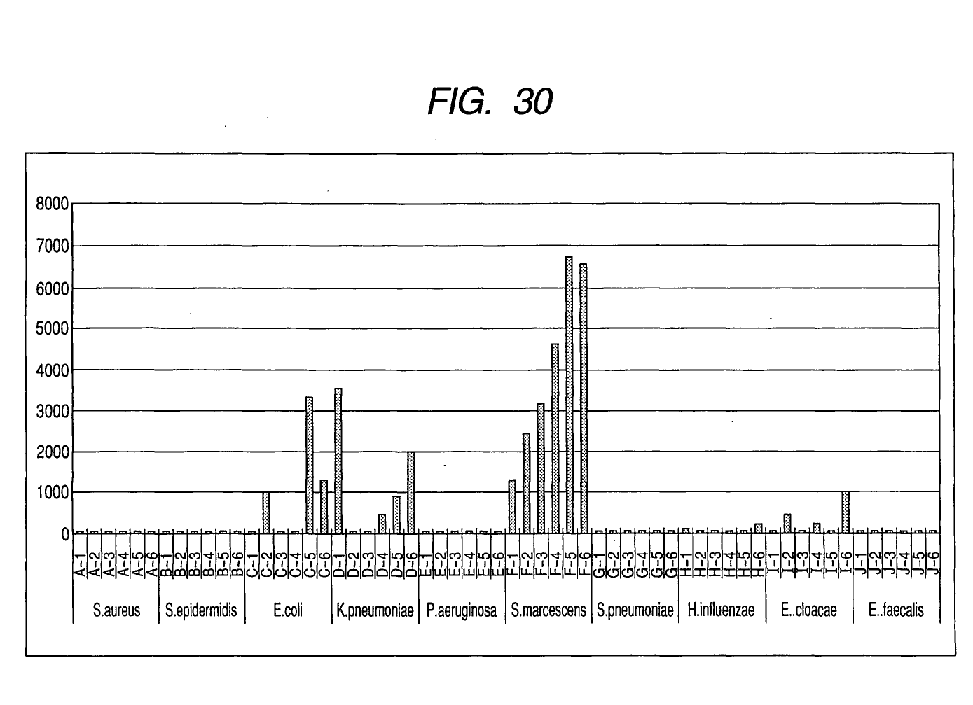

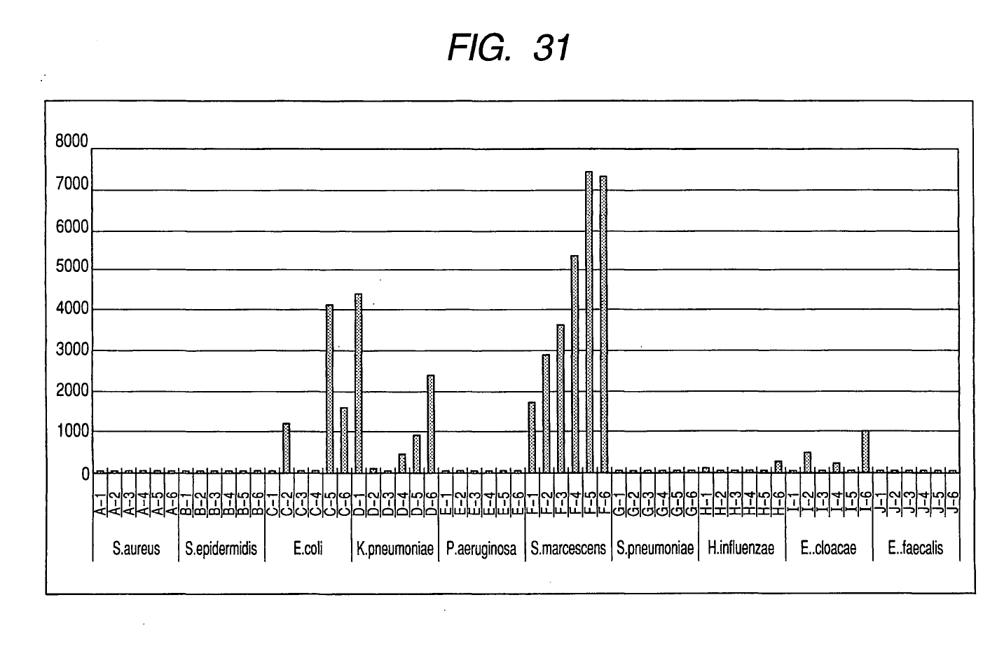

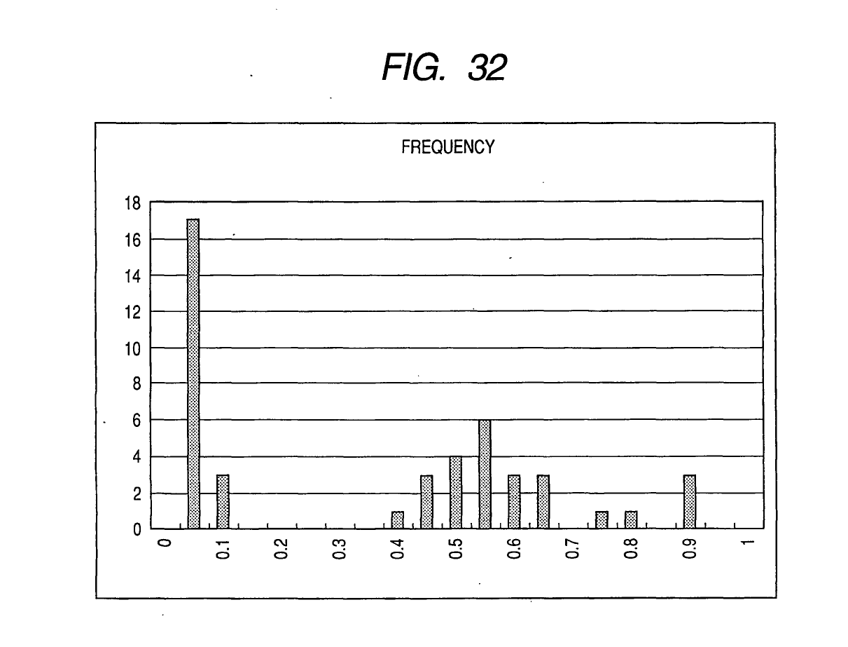

Next, experimental data on 10 samples of S. marcescens are similarly represented in FIGS. 22 to 31. Using the normalization and distance calculation carried out for K. pneumoniae, the distance between two arbitrary samples of 10 samples is calculated and then represented as a histogram as shown in FIG. 32. It is found that the outline of each of the histogram and distribution of K. pneumoniae is completely different from others. The presence of two large peaks assumes that there are two clusters in 10 vectors. Actually, it is found that there are roughly two different patterns as is also evident from the fluorescence intensity graph of 10 samples as previously described above. From this figure, when the determination is conducted by applying the above k-th nearest neighbor algorithm on K. pneumoniae, one of the candidates of a critical value thereof is the maximum value, 0.090, of the first peak.

The present invention is not limited to the above embodiments and various changes and modifications can be made within the spirit and scope of the present invention. Therefore to apprise the public of the scope of the present invention, the following claims are made.

This application claims priority from Japanese Patent Application No. 2005-227995 filed Aug. 5, 2005, which is hereby incorporated by reference herein in its entirety.

1. A method of determining a biospecies by analyzing a sample, in which a substance derived from an organism is supposed to be included, to determine the biospecies corresponding to the organism, comprising the steps of:
obtaining a plurality of analysis data by analyzing a plurality of known samples whose corresponding biospecies are already revealed, by a method of analyzing a biospecies;
defining a determination threshold with respect to the biospecies corresponding to the known sample on the basis of the plurality of analysis data obtained from the plurality of known samples;
obtaining analysis data for specifying a biospecies corresponding to an unknown sample whose corresponding biospecies is unknown, by analyzing the unknown sample by the method of analyzing a biospecies;
deciding whether determination of a species corresponding to the unknown sample is possible or impossible on the basis of the determination threshold; and
determining the biospecies of the unknown sample on the basis of the plurality of analysis data when the determination is decided as possible.

2. A method of determining a biospecies according to claim 1, wherein the determination threshold is defined by removing an arbitrary analysis data from a total set of the plurality of analysis data obtained from the known samples for one biospecies and stored in a storage means, creating an identification dictionary composed of learnings on the basis of the remaining analysis data, determining the removed analysis data on the basis of the identification dictionary to induce a determination index, and obtaining the determination threshold on the basis of the determination index.

3. A method of determining a biospecies by analyzing a sample, in which a substance derived from an organism is supposed to be included, using a method of analyzing a biospecies to determine the biospecies corresponding to the organism, comprising the steps of:
(1) selecting a biospecies to be supposed as a result of determination with respect to an unknown sample;
(2) obtaining an assembly of image data composed of a plurality of image data specific to the biospecies and usable for pattern recognition from each of known samples obtained from a plurality of individuals which are already revealed to belong to the selected biospecies;
(3) defining a determination threshold by selecting image data from the assembly of image data and using a relationship with the remaining image data;
(4) obtaining image data from an unknown sample;
(5) determining whether determination of a species corresponding to the unknown sample is possible or impossible on the basis of the determination threshold with respect to the image data from the unknown sample; and
(6) determining the biospecies using an identification dictionary comprising the assembly of image data when the determination is decided as possible in the step (5).

4. A method of determining a biospecies according to claim 3, wherein the step of defining a determination threshold includes the steps of:
(1) obtaining an assembly of image data composed of three or more image data obtained by selecting three or more different individuals as the individuals;
(2) carrying out a process on each of the image data and obtaining a determination index set composed of “m” determination indices, the process including selecting and removing one image data from the assembly of image data, creating a dictionary by using the remaining plurality of image data, and obtaining a determination index by determining the image data previously removed on the basis of the obtained dictionary; and
(3) defining the determination threshold from the determination index set.

5. A method of determining a biospecies according to claim 3, wherein the step of defining a determination threshold includes the steps of:
(1) obtaining an assembly of image data composed of three or more image data obtained by selecting three or more different individuals as the individuals;
(2) obtaining a distance set by obtaining a distance between image data on two individuals with respect to every combination of image data on two arbitrary individuals selected from the assembly of image data; and
(3) defining the determination threshold from the distance set.

6. A method of determining a biospecies according to claim 3, wherein the method of determining a biospecies comprises a method of obtaining image data, which includes the steps of:
causing a nucleic acid sample serving as a known or unknown sample to react with a probe-immobilizing carrier, in which a probe capable of specifically binding to a target nucleic acid having a nucleic acid sequence specific to the selected organism is immobilized on a predetermined position on a substrate; and
optically detecting a hybrid of the target nucleic acid and the probe formed on the substrate.

7. A method of determining a biospecies according to claim 6, wherein the step of optically detecting a hybrid includes utilizing fluorescence from a fluorescent label attached on the hybrid.

8. An information processing apparatus for determining a biospecies, comprising:
a memory for storing a plurality of analysis data obtained by analyzing a plurality of known samples whose corresponding biospecies are already revealed by a method of analyzing an organism, and a determination threshold defined on the basis of the plurality of analysis data; and
a processing unit for deciding whether determination a biospecies corresponding to an unknown sample is possible or not, and determining the biospecies corresponding to the unknown sample on the basis of the plurality of analysis data stored in the memory when the determination is decided as possible.

9. An information processing apparatus for determining a corresponding biospecies by analyzing a sample in which a substance derived from an organism is supposed to be included, comprising:
a known sample image data inputting means for inputting image data specific to the biospecies obtained by analyzing known samples from a plurality of individuals whose corresponding biospecies are already revealed;
an unknown sample image data inputting means for inputting image data obtained by analyzing the unknown sample in a similar manner to a case of the known sample;
a first storage means for storing the image data captured;
means for defining a determination threshold with respect to a biospecies corresponding to the known sample on the basis of the plurality of analysis data obtained from the known samples;
a biospecies determining means for deciding whether the determination is possible or not on the basis of the determination threshold with respect to image data from the unknown sample, and determining the biospecies corresponding to the unknown sample when the determination is decided as possible;
a second storage means for storing a determination result obtained by the biospecies determining means; and
an output means for outputting the determination result stored in the second storage means.

10. An information processing apparatus according to claim 9, wherein:
the number of the individuals is equal to or greater than three;
the image data from the individuals are stored in the first storage means; and
the determination threshold is defined on the basis of a program including the steps of:
(a) carrying out a process on each of the image data and obtaining a determination index set composed of three or more determination indices, the process including selecting and removing image data on one individual from image data on the three or more individuals, creating an identification dictionary using image data on the remaining plurality of individuals, and obtaining a determination index by determining the image data previously removed on the basis of the obtained identification dictionary; and
(b) defining the determination threshold from the determination index set.

11. An information processing apparatus according to claim 9, wherein:
the number of the individuals is equal to or greater than three;
the image data from the individuals are stored in the first storage means; and
the determination threshold is defined on the basis of a program having the steps of:
(A) obtaining a distance set by obtaining a distance between image data on two individuals with respect to every combination of image data on two arbitrary individuals selected from image data on the three or more individuals; and
(B) defining the determination threshold from the distance set.

12. A program for determination of a biospecies, for causing a computer to execute determination of a biospecies corresponding to an unknown sample, comprising the steps of:
(1) calling a plurality of known sample image data from a first storage means that stores a plurality of image data corresponding to image data specific to a biospecies to be supposed as a result of determination with respect to the unknown sample obtained by analyzing known samples from a plurality of different individuals belonging to the biospecies to be supposed;
(2) reading out the unknown sample image data from the first storage means for storing a plurality of image data corresponding to image data obtained by analyzing the unknown sample in a similar manner to a case of the known samples;
(3) defining a determination threshold by selecting one of the unknown sample image data and utilizing a relationship between the selected one and the remaining image data;
(4) determining a species of an organism corresponding to the unknown sample by processing the unknown sample image data on the basis of the determination threshold;
(5) storing a determination result obtained in the determination step into a second storage means; and
(6) outputting the determination result stored in the second storage means.

13. A program for determination of a biospecies according to claim 12, wherein:
the number of the individuals is equal to or greater than three;
the image data from the individuals are stored in the first storage means; and
the step of determining a species includes the steps of:
(a) carrying out a process on each of the image data and obtaining a determination index set composed of three or more determination indices, the process including selecting and removing image data on one individual from image data on the three or more individuals, creating an identification dictionary using image data on the remaining plurality of individuals, and obtaining a determination index by determining the image data previously removed on the basis of the obtained identification dictionary; and
(b) defining the determination threshold from the determination index set.

14. A program for determination of a biospecies according to claim 12, wherein:
the number of the individuals is equal to or greater than three;
the image data from the individuals are stored in the first storage means; and
the step of determining a species includes the steps of:
(A) obtaining a distance set by obtaining a distance between image data on two individuals with respect to every combination of image data on two arbitrary individuals selected from image data on the three or more individuals; and
(B) defining the determination threshold from the distance set.

15. A recording medium, which is recorded in a readable manner with a program for causing a computer to execute determination of a biospecies, wherein the program comprises the program according to claim 12.

16. A method of determining a biospecies, comprising:
using a plurality of analysis data obtained by analyzing a plurality of known samples whose corresponding biospecies are already revealed by a method of analyzing an organism and a determination threshold defined on the basis of the plurality of analysis data;
deciding whether determination of a biospecies corresponding to an unknown sample is possible or not on the basis of the determination threshold; and
determining a biospecies corresponding to the unknown sample on the basis of the plurality of analysis data when the determination is decided as possible.
